(19) United States
(12) Patent Application Publication
(10) Pub. No.: US 2015/0323541 A1
(43) Pub. Date: Nov. 12, 2015

(54) METHODS AND REAGENTS FOR ANALYTE DETECTION

(71) Applicant: ORANGENE INC., Frederick, MD (US)

(72) Inventor: Qun LIU, Boyds, MD (US)

(73) Assignee: ORANGENE INC., Frederick, MD (US)

(21) Appl. No.: 14/803,600

(22) Filed: Jul. 20, 2015

Related U.S. Application Data
(63) Continuation of application No. 12/924,072, filed on Sep. 20, 2010.
(60) Provisional application No. 61/276,954, filed on Sep. 19, 2009.

(51) Int. Cl. G01N 33/58; C12Q 1/68

(52) U.S. Cl. CPC G01N 33/581; C12Q 1/6818; G01N 33/587

(57) ABSTRACT

The present invention relates to chemiluminescent method and regent to detect analyte. One aspect of the current invention relates to using chemiluminescent and fluorescent molecule/enzyme coupled with analyte binding molecules to detect specific analyte molecules. Another aspect of the current invention is to use gold nanoparticle triggered chemiluminescent reaction to detect analyte.

### CROSS-REFERENCE TO RELATED APPLICATION

This application claims priority to U.S. Provisional Patent Application No. 61/276,954 filed on Sep. 19, 2009, and is a continuation of U.S. Ser. No. 12/924,072, filed Sep. 20, 2010. The entire disclosures of the prior applications is considered to be part of the disclosure of the instant application and is hereby incorporated by reference.

### TECHNICAL FIELD

The present invention relates to chemiluminescent methods and regents to detect analyte. One aspect of the current invention relates to using chemiluminescent and fluorescent molecule/enzyme coupled with analyte binding molecules to detect specific analyte molecules. Another aspect of the current invention is to use gold nanoparticle triggered chemiluminescent reaction to detect analyte.

### BACKGROUND OF THE INVENTION

Chemiluminescence (CL, sometimes “chemoluminescence”) is the emission of light (luminescence) with limited emission of heat as the result of a chemical reaction. Light-emitting systems have been known and isolated from many luminescent organisms, including certain bacteria, protozoa, coelenterates, mollusks, fish, millipedes, flies, fungi, worms, crustaceans, and beetles. Those enzymes isolated from beetles, particularly the fireflies of the genera Photinus, Photuris and Luciola and click beetles of genus Pyrophorus have found widespread use in reporter systems. In many of these organisms, enzymatically catalyzed oxidoreductions take place in which the free energy change is utilized to excite a molecule to a high-energy state. When the excited molecule spontaneously returns to the ground state, visible light is emitted. This emitted light is called “bioluminescence” or “chemoluminescence”. Firefly luciferase or click beetle luciferase catalyses the oxidation of firefly luciferin in the presence of ATP, Mg2+ and molecular oxygen with the resultant production of light. There are also other types of luciferin that can trigger luminescent reaction. Bacterial luciferin is a reduced riboflavin phosphate (FMNH2), which is oxidized in association with a long-chain aldehyde, oxygen, and a bacterial luciferase. Dinoflagellate luciferin is derived from chlorophyll, and has a very similar structure. In the genus Gonyaulax, at pH 8 the molecule is “protected” from the luciferase by a “luciferin-binding protein”, but when the pH lowers to around 6, the free luciferin reacts and light is produced. Vargulin is found in the ostracod (“seed shrimp”) Vargula, and is also used by the midshipman fish Porichthys. Coelenterazine is the most “popular” of the marine luciferins, found in a variety of phyla. This molecule can occur in luciferin-luciferase systems, and is famous for being the light emitter of the photoprotein “aequorin”. Besides enzyme-catalyzed chemiluminescence, small organic molecule based chemiluminescence (CL) assays are also widely used for analyte detection. The most important chemiluminescent compounds include luminol, acridinium, acridan and 1,2-dioxetane.

### SUMMARY OF THE INVENTION

The present invention relates to chemiluminescent methods and regents to detect analyte. One aspect of the current invention relates to using chemiluminescent molecule/enzyme coupled with analyte binding molecules to detect specific analyte molecules involving chemiluminescent (bioluminescent) binding pair-fluorescent binding pair system. Another aspect of the current invention is to use gold nanoparticle triggered chemiluminescent reaction to detect analyte. The gold nanoparticle aggregate upon analyte binding therefore change its capability to induce chemiluminescence. The changed chemiluminescence enables the presence and amount of the analyte.

### BRIEF DESCRIPTION OF THE DRAWINGS

FIG. 1 is the principle of detecting analyte using a chemiluminescent (bioluminescent) binding pair-fluorescent binding pair system.

### DETAILED DESCRIPTION OF THE INVENTIONS

There are several general types of luciferin: Firefly luciferin is the luciferin found in firefly. It is the substrate of firefly luciferase. Bacterial luciferin is a type of luciferin found in bacteria, some squid and fish. It consists of a long-chain aldehyde and a reduced riboflavin phosphate. Dinoflagellate luciferin is a chlorophyll derivative and is found in dinoflagellates. A very similar type of luciferin is found in some types of euphausiid shrimp. Another luciferin called vargulin is found in certain deep-sea fish, specifically, in ostracods and poricthys. The fifth luciferin called coelenterazine is found in radiolarians, ctenophores, cnidarians, squid, copepods, chaetognaths, fish and shrimp. It is the light-emitting molecule in the protein called aequorin. Yet another luciferin is called latia luciferin, which can be found in sea latia neritoides. In the current inventions described above and below, the term firefly luciferase include both the native firefly luciferase extracted from firefly and those engineered firefly luciferase such as those generated by mutation for better thermal stability or different optimum pH or emission wavelength. There are many engineered firefly luciferase that can be found in scientific publications and patents and many of them are commercially available. Any firefly luciferase is suitable for the current invention as long as it uses firefly luciferin or firefly luciferin directives (e.g. 6-amino firefly luciferin) for luminescence. Since there are also other luciferases that utilize firefly luciferin to emit light, for example, the click beetle luciferase, these luciferases can also be used to replace the firefly luciferase used in the current invention.

One aspect of the current invention involves the use of the potential chemiluminescent compound-enzyme substrate conjugate to detect the presence of target enzyme. The enzyme breaks the conjugate and releases the potential chemiluminescent compound. The chemiluminescent compound can emit detectable light under suitable conditions and therefore indicate the presence of certain target enzyme. The chemiluminescent compounds used in the current invention are firefly luciferin or its analogue. The enzyme can be detected include but not limited to alpha-L-Arabinosidase, beta-Cellobiosidase, alpha-L-Fucosidase, beta-D-fucosidase, beta-L-Fucosidase, alpha-Galactosaminidase, beta-Galactosaminidase, alpha-Galactosidase, beta-Galactosidase, alpha-Glucosaminidase, beta-Glucosaminidase, neuraminidase, proline aminopeptidase, leukocyte esterase, alpha-L-fucosidase, glycylproline dipeptidyl aminopeptidase, beta-galactosaminidase, N-acetyl-beta-D-glucosaminidase, Salmonella esterase, beta-glucuronidase and hydroxyproline aminopeptidase. For example, the conjugate of N-acetylneuraminic acid and firefly luciferin is substrate for neuraminidase, which cleaves the substrate to give rise to free firefly luciferin that is the substrate of luciferase, which is present in the detection mix.

The conjugate itself is not a substrate for firefly luciferase. Therefore, the luciferase-catalyzed biochemiluminescence is dependent on neuraminidase activity, which is provided by the influenza virus or other organisms to be detected. Further more, the same principle also works for other enzyme when suitable chemiluminescent substrate is used as long as the substrate can be cleaved by the target enzyme and the cleavage product can emit light for detection.

It has been reported that different firefly luciferin derivative can emit light at different wavelength. Therefore using these luciferin derivatives to construct substrate for different enzymes will allow the different enzyme causing different wavelength of light emitting. Therefore using these substrates in one assay mix will allow multiplex detection for multiple enzymes simultaneously. For example, 3 luciferin emitting different wavelength of light (e.g. 450 nm, 560 nm and 680 nm) can be coupled with 3 different peptide therefore allow the detection of different peptidase at the same time based on their different emitting wavelength. If the —NMe2 group is replaced with —OH group, they can be used to couple with different sugars to detect different glycosidase in a multiplex assay.

However, sometimes the release of luciferin from the conjugate is slow or not possible for some enzyme. Therefore, using precursor of luciferin instead of luciferin to form the conjugate as these enzyme's substrate will be suitable in these applications. The released precursor can react with other compound to form luciferin, which can generate chemiluminescence (CL) for detection. Examples of luciferin precursor include 2-cyano-6-hydroxybenzothiazole and 2-Cyano-6-aminobenzothiazole, also called 6-amino-2-benzothiazolecarbonitrile (CAS No. 7724-12-1), which can react with D-cysteine to generate luciferin for detection. The formula I below shows an example of the substrate using the 2-cyano-6-hydroxybenzothiazole for neuraminidase detection.

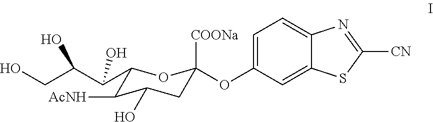

The assay can be done in one step or two-step fashion. The two-step assay separates the enzyme cleavage step with the CL step. The key feature of one-step method is the combination of target enzyme (e.g. neuraminidase) reaction with the generation of luciferin from its precursor and the CL reaction in a single step. In this assay format, luciferin is formed and detected as it's precursor is being released through the action of target enzyme (e.g. neuraminidase) and the luciferin is formed by react the released precursor with D-cysteine. This method is therefore referred to as real time detection of target enzyme (e.g. neuraminidase) or real time detection method. In the real time enzyme detection method, the detection mix contains all necessary chemicals and appropriate buffer for target enzyme reaction, including the conjugate itself, and for luciferase-catalyzed CL reaction except for luciferin. In the current inventions, the term chemiluminescence and bioluminescence are used interchangeable. Luciferin and luciferase are not specific molecules. They are generic terms for a substrate and its associated enzyme (or protein) that catalyze a light-producing reaction. A variety of species regulate their light production using different luciferases in a variety of light-emitting reactions. Luciferins are a class of small-molecule substrates, each being specific for its corresponding protein enzyme luciferase. Luciferins are catalyzed in the presence of the enzyme luciferase to produce light.

In one embodiment, the influenza test kit contains two key components: conjugate mix and detection mix. It is used for detection of influenza neruaminidase.

Reagent Compositions

Conjugate Mix 1 (lyophilization is preferred)

| MES, pH 6.5 | 32.5 | mM |
|---|---|---|
| CaCl₂ | 4 | mM |
| BSA | 1 | mg/mL |
| Triton X-100 | 0.5% | |
| Mannitol | 4% | |
| Sucrose | 1% | |
| Conjugate substrate | 100 | μg/mL |

| | |
|---|---|
| D-cysteine | 1% |

Detection Mix 1 (lyophilization is preferred)

| Imidazole, pH 7.8 | 100 | mM |
|---|---|---|
| MgSO₄ | 15 | mM |
| BSA | 1 | mg/mL |
| ATP, Na Salt | 12 | mM |
| DTT | 10 | mM |
| Co-enzyme A | 1 | mM |
| Mannitol | 4% | |
| Sucrose | 1% | |
| Firefly Luciferase | 20 | μg/mL |

(or Click beetle luciferase)

The D-cysteine can also be placed in the detection mix 1 instead.
The influenza virus detection assay comprises essentially two steps: 1) cleavage of sialic acid-firefly luciferin precursor conjugate with influenza neuraminidase, and 2) detection of released firefly luciferin precursor by detecting the luciferin generated from it with D-cysteine. Specifically one can use the following basic protocol:
1. Mix the sample containing flu virus with 100 μL conjugate solution, which will lyse the virus because of the presence of Triton X-100.
2. Incubate at room temperature for 10-15 minutes.
3. Transfer the solution to 100 μL firefly luciferase detection solution pre-loaded into a detection tube, or a lyophilized detection mix in a detection tube.
4. Place the detection tube into a luminomter and record the light signal (relative light unit) for an appropriate period of time (e.g., 5 min).

In another embodiment, the assay is performed in one step format using detection mix 2. In brief, influenza virus collected in the throat nasal swab is lysed in a virus lysis buffer (PBS+1% Triton X 100). A portion of the lysis buffer (200 μL) is then added to a pre-mix containing all necessary reagents, followed by incubation for 10-15 minutes at room temperature. Presence of influenza virus, hence the viral neuraminidase, results in the cleavage of a substrate, which enables the generation of luciferin and then visible light signal that is detected with a portable luminometer. Click beetle luciferase can also be used instead of the firefly luciferase.

Detection Mix 2 (lyophilized form preferred)

| Imidazole, pH 7.0-7.2 | 50 | mM |
|---|---|---|
| BSA | 1 | mg/mL |
| ATP, Na Salt | 12 | mM |
| DTT | 10 | mM |
| Co-enzyme A | 1 mM or 10 mg/mL | |
| MgSO4 | 15 | mM |
| CaCl2 | 4 | mM |
| Mannitol | 4% | |
| Sucrose | 1% | |
| Sodium Azide | 0.05% | |
| Firefly Luciferase | 20 | μg/mL |
| Conjugate substrate | 100 | μg/mL |

| | |
|---|---|
| D-cysteine | 1% |

Assay Protocol

Step 1—Sample Preparation:
Place the throat swab into the Virus Lysis Buffer tube,
Roll the swab at least three times while against the bottom and side of the tube,
Wring out the swab by squeezing the tube wall against the swab and carefully pulling out the swab from the tube.
Discard the swab in a biohazard container.
Step 2—Transfer 200 μL of the sample prepared in Step 1 to a Detection Mix test tube. Cap the vial. Gently swirl the tube until all of the lyophilized powder is wet. Particulate materials may initially be present in the mix, which does not interfere with the detection.
Step 3—Incubate at room temperature (20-30° C.) for 15 minutes.
Step 4—Place the test tube in the luminometer and press the start button. Record and print the test results.

If the assay involves the detection of 6-amino firefly luciferin, the 2-Cyano-6-aminobenzothiazole should be used to form the substrate. For example, elevated proline aminopeptidase activity in vaginal fluid has been associated with bacterial vaginosis. Thus, a proline aminopeptidase assay can also be used to diagnose bacterial vaginosis. Proline aminopeptidase (or called proline iminopeptidase) is a hydrolase that cleaves the L-proline residues from the N-terminal position in peptides. A substrate that can be used in a proline aminopeptidase assay for bacterial vaginosis diagnosis is L-proline-6-amino firefly luciferin precursor conjugate (L-prolyl-2-Cyano-6-aminobenzothiazole) or its derivatives. In a proline aminopeptidase assay, the enzyme cleaves the synthetic substrate and releases the free 2-Cyano-6-aminobenzothiazole which can be converted to 6-amino firefly luciferin by react with D-cysteine, which can be quantitatively detected in a CL reaction in the presence of firefly luciferase and ATP. It has the following formula II.

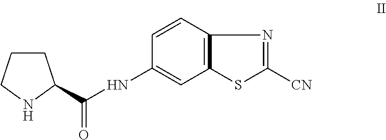 [SMILES: N#Cc1nc2ccc(NC(=O)[C@@H]3CCCN3)cc2s1]

In one embodiment, the sample is mixed with 0.1 ml of L-proline aminopeptidase detection mix (3 mg of substrate above dissolved in 1 mL of 0.5 M sodium acetate buffer at a pH of about 8.0) in a vial, incubated for 10 minutes 30 degree C., then add 0.1 ml 1% D-cysteine in 0.1 M PBS buffer and incubate for another 10 min, then mixed with 100 ul detection mix 1 described above and measure the light signal using a luminometer. Interpretation of the test result (positive or negative for aminopeptidase) is based on the established cutoff value.

Below are more examples of the substrate using the firefly luciferin precursor methods:

| Sub- | | |
|---|---|---|
| stitution | | |
| symbol | Substitution group | Substrate for enzyme |
| Rs | -alpha-L-arabinopyranoside | alpha-L-Arabinosidase |
| Rs | -beta-D-cellobioside | beta-Cellobiosidase |
| Rs | -alpha-L-fucopyranoside | alpha-L-Fucosidase |
| | (Described in the above AFU | |
| | assay) | |
| Rs | -beta-D-fucopyranoside | beta-D-fucosidase |
| Rs | -beta-L-fucopyranoside | beta-L-Fucosidase |
| Rs | -N-acetyl-alpha-D- | alpha-Galactosaminidase |
| | galactosaminide | |
| Rs | -N-acetyl-beta-D-galactosaminide | beta-Galactosaminidase |
| | (for Candida albicans test) | |
| Rs | -alpha-D-galactopyranoside | alpha-Galactosidase |
| Rs | -beta-D-galactopyranoside | beta-Galactosidase |
| Rs | -N-acetyl-alpha-D-glucosaminide | alpha-Glucosaminidase |
| Rs | -N-acetyl-beta-D-glucosaminide | beta-Glucosaminidase |
| | (for NAGase test) | |
| Rs | -alpha-D-glucopyranoside | alpha-Glucosidase |
| Rs | -beta-D-glucopyranoside | beta-Glucosidase |
| Rs | -beta-D-glucuronic acid | beta-Glucuronidase |
| | (for beta-Glucuronidase test) | |
| Rs | -beta-D-lactopyranoside | beta-Lactosidase |
| Rs | -beta-D-maltopyranoside | alpha-Maltosidase |
| Rs | -alpha-D-mannopyranoside | alpha-Mannosidase |
| Rs | -beta-D-mannopyranoside | beta-Mannosidase |
| Rs | -beta-D-xylopyranoside | beta-Xylosidase |

Similar to the substrate used in the sialidase assay, the Rs is the 2-cyano-6-hydroxybenzothiazole conjugated to the sugar, which has the following formula III:

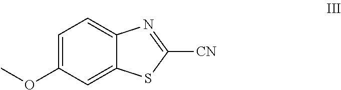

If the Rs is firefly luciferin itself, it will be the original (none precursor) substrate.

Elevated glycylproline dipeptidyl aminopeptidase (GPDA) activity in blood and urine is associated with abnormality in liver, stomach, intestine and kidney. In one example, The GPDA assay is based on the enzymatic cleavage of the synthetic substrate L-glycyl-L-prolyl-2-Cyano-6-aminobenzothiazole (similar to the L-prolyl-2-Cyano-6-aminobenzothiazole described above). In the GPDA assay, the L-glycyl-L-prolyl-6-amino firefly luciferin precursor conjugate is cleaved by GPDA to give rise to free luciferin which is formed by reacting with D cysteine in the reagent mix, which is detected in a firefly luciferase catalyzed CL reaction. It can be a one-step assay or two steps assay as described before. In another example, the following substrate having the formula IV is used for the esterase such as acetylcholine esterase detection:

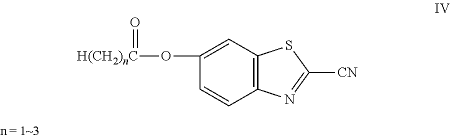

The synthesis of substrate containing 6-amino firefly luciferin such as peptide 6-amino firefly luciferin conjugate normally is done by coupling peptide or other molecule with 6-amino firefly luciferin using solution phase coupling method, e.g. fragment condensation method. This method has the problem of low yield and difficult to couple and purify. Here a new method to synthesize substrate containing 6-amino firefly luciferin or its directive is disclosed. The method generally involves the following steps. First 6-amino firefly luciferin is immobilized on the solid phase support, then the standard peptide synthesis or organic synthesis is performed on the solid phase support to complete the synthesis of the conjugate, then the product is cleaved from the solid phase support to release the resulting desired final product. Alternatively, D-cysteine is coupled to the solid phase support, next the 2-Cyano-6-aminobenzothiazole is react with the immobilized—cysteine to generate the 6-amino firefly luciferin on the solid phase support, then the standard peptide synthesis or organic synthesis is performed on the solid phase support to complete the synthesis of the conjugate, then the product is cleaved from the solid phase support to release the resulting desired final product.

For example, in order to synthesis L-glycyl-L-prolyl-6-amino firefly luciferin, Fmoc protected 6-amino firefly luciferin is react with NovaPEG Wang resin using standard amino acid immobilization chemistry, after the Fmoc protected 6-amino firefly luciferin is coupled to the resin, the Fmoc group is cleaved using standard piperidine deprotection chemistry. The free amine is exposed. Next the Fmoc Proline and Fmoc-glycine is coupled to the resin stepwise using standard Fmoc peptide coupling chemistry. Next the product is cleaved with standard TFA chemistry (e.g. 20-90% TFA in DCM) to release the resulting L-glycyl-L-prolyl-6-amino firefly luciferin. Alternatively, one can use Rink Acid resin or NovaPEG HMPB resin or 2-chlorotrityl resins to immobilize the 6-amino firefly luciferin or Fmoc protected 6-amino firefly luciferin at the first step, and the final step can be achieved with much weaker acid such as 1% TFA in DCM or 10% AcOH in DCM to cleave the product from the resin.

Alternatively, D-cysteine is coupled to the solid phase support first, for example, one can use Fmoc-D-Cys(Acm)-OH (The side-chain Acm group can be removed with Hg(II) or Ag(I)) or Fmoc-D-Cys-OH or Fmoc-D-Cys(Mmt)-OH (The Mmt group can be deprotected on the solid phase with 1% TFA in DCM containing 5% TIS) or Fmoc-D-Cys(tButhio)-OH (The t-Buthio group can be selectively removed using thiols or tributylphosphine) to couple with suitable resin such as NovaPEG Wang resin or Rink Acid resin or NovaPEG HMPB resin or 2-chlorotrityl resins. After the protected D-cysteine is immobilized, the Fmoc group and the side chain-protecting group (if any) are removed. Now the resin is D-cysteine-resin with amino and thiol group free. Next it is react with the 2-Cyano-6-aminobenzothiazole in DMSO/water 1:1 pH=7.5 for 2 hours. The resulting product is 6-amino firefly luciferin on the solid phase resin, which is ready for further coupling.

The United States Patent Application 20070264664 “Nonseparation assay methods” described non-separation assay methods. The disclosed assay methods involve a compound capable of producing CL which is immobilized on a solid support as a member of a specific binding pair for capturing an analyte from a sample; an activator compound that activates the chemiluminescent compound conjugated to a specific binding pair member is added in excess along with the sample to the solid support. Addition of a trigger solution causes a chemiluminescent reaction at the sites where the activator conjugate has been specifically bound. The assay methods are termed non-separation assays because they do not require removal or separation of excess detection label (activator conjugate) prior to the detection step. The methods in 20070264664 application are applicable to various types of assays including immunoassays, receptor-ligand assays and nucleic acid hybridization assays. The methods in 20070264664 application feature the use of an immobilized chemiluminescent compound and an activator compound conjugated to a specific binding partner for inducing a chemiluminescent reaction. Analyte-mediated co-localization of the chemiluminescent label compound and the activator conjugate causes the ensuing chemiluminescent reaction to take place only at the site of the bound analyte molecules. The presence of unbound, excess activator conjugate does not contribute to or interfere with the chemiluminescent reaction. As a result, the intensity of CL emitted is proportional to the quantity of analyte.

Assays performed according to the 20070264664 application involve four steps. In a first step a solid phase is provided in a test device for specifically capturing an analyte of interest. The solid phase is provided with an immobilized specific binding partner for an analyte to be detected. The solid phase is further provided with chemiluminescent labeling compound immobilized thereon. The chemiluminescent label may be provided in a number of different ways as described in the 20070264664 application. In each variant the chemiluminescent label is irreversibly attached to a substance or material in a way that renders it immobile. In a second step the analyte-containing sample and the activator conjugate are introduced to the test device having the solid phase immobilized specific binding partner for the analyte and permitted to form specific binding complexes. They can be added separately in either order, or simultaneously, or can be pre-mixed and added as a combination. An optional delay time to allow binding reactions to occur can be inserted at this point. In the third step a triggers solution is added to produce the CL for detecting the analyte. Lastly the chemiluminescence is detected. Preferably either peak light intensity level or total integrated light intensity is measured. The quantity of light can be related to the amount of the analyte by constructing a calibration curve according to generally known methods. When light emission ensues rapidly after addition of trigger solution it is desirable to either mechanically time the onset of measurement to the addition by means of a suitable injector or to perform the addition with the test device already exposed to the detector.

The activator compound in 20070264664 application forms part of an activator-specific binding partner conjugate. The conjugate serves a dual function; 1) binding specifically to the analyte in the assay through the specific binding partner portion, either directly or through an intermediary specific binding partner, and 2) activating the chemiluminescent compound through the activator portion. The activator portion of the conjugate is a compound that effects the activation of the chemiluminescent compound so that, in the presence of the trigger solution, CL is produced. Compounds capable of serving as the activator include transition metal salts and complexes and enzymes, especially transition metal-containing enzymes, most especially peroxidase enzymes. Transition metals useful in activator compounds include those of groups 3-12 of the periodic table, especially iron, copper, cobalt, zinc, manganese, and chromium. It should be noted that the activator molecules responsible for signal generation may operate within a physically confined radius and only have contact with a finite supply of chemiluminescent compound. This would seem to preclude large catalytic turnover in cases where the activator possesses that potential. The peroxidase which can undergo the chemiluminescent reaction include lactoperoxidase, microperoxidase, myeloperoxidase, haloperoxidase, e.g. vanadium bromoperoxidase, horseradish peroxidase, fungal peroxidases such as lignin peroxidase and peroxidase from Arthromyces ramosus and Mn-dependent peroxidase produced in white rot fungi, and soybean peroxidase. Other peroxidase mimetic compounds which are not enzymes but possess peroxidase-like activity including iron complexes, such as gold nano particle, heme, and Mn-TPPS₄ are known which catalyze the chemiluminescent oxidation of substrates are explicitly considered to be within the scope of the meaning of peroxidase as used herein. The activator produces highly active oxygen and/or active radical which can be consumed by the nearby chemiluminescent compound to generate detectable CL signal. However, not only the activator which had already formed binding complex with the analyte and the chemiluminescent compound produce highly active oxygen and/or active radical at the presence of trigger solution; in fact, all the activator compounds including the those in the unbound form produce highly active oxygen. These highly active oxygen and active radical will also be consumed by chemiluminescent compound in the non bound form and generate light signals. These light signals are not related to the presence of analyte therefore become background (noise) signal in the assay.

The current invention provides a method to improve the performance of none separation assay described in the 20070264664 application by increase the signal/background ratio of the assay. The signal in the signal/noise(background) ratio is produced by the analyte. The definition of signal/noise(background) ratio is well known to the skilled in the art. The principle is by adding highly active oxygen/active free radical scavenger such as antioxidant/free radical scavenger (e.g., VC, VE, BHT and Cysteine) or enzymes that can decompose the highly active oxygen, e.g. superoxide dismutase (SOD, e.g. 58160 or 55389 from Sigma) to the assay mix, the background will decrease. But in the bound form because the highly active oxygen/radical is produced very close to the chemiluminescent compound, it has higher chance to produce light than being consumed by antioxidant/free radical scavenger/decomposing enzymes. Therefore the signal produced by analyte is less likely to be decreased. There are many antioxidant/free radical scavenger/reductant can be used for the assay, examples of them include glutathione, vitamin C, and vitamin E, coenzyme Q, thiol containing compounds, melatonin, thiols or polyphenols (e.g. tea polyphenol). The skilled in the art can find many of them and the optimal one or combination thereof can be found by screening them in the assay for the highest signal/noise ratio. But one need to know that there are several different types of active oxygen/radical such as superoxide anion and hydroxyl radical. Different CL compounds may be triggered by different type of active oxygen therefore they may require different antioxidant/free radical scavenger/reductant for the assay. For example, SOD works fine for the luminol system while catalase works for the acridan system.

The optimal type and amount need to be added to the reaction mix can be determined experimentally, in general, the assay is performed according to the protocol described in 20070264664 application, different amount of antioxidant/free radical scavenger/enzyme that can decompose the highly active oxygen are added to the assay mix (e.g. immediately before the adding of the trigger solution) and the amount giving the best signal/noise ratio is selected. For example, in order to screen the optimal concentration of antioxidant VC or TP (tea polyphenol) or SOD, a series of concentration of VC or TP or SOD (make their final concentration in the assay mix ranging from 1 ng/ml, 10 ng/ml, 100 ng/ml, 1 ug/ml, 10 ug/ml, 100 ug/ml, 1 mg/ml, 10 mg/ml) can be added to the assay mix and the assay is performed, the concentration giving the best signal/noise ratio will be selected and further optimized. For example, if 10 ug/ml give the best result, 2 ug/ml, 5 ug/ml, 10 ug/ml, 20 ug/ml, 50 ug/ml can be further tested to see if better result can be obtained. Also, this method can allow the chemiluminescent compound conjugated to a specific binding pair member directly instead of immobilized on the solid support, e.g. in the example 1 described below, the acridan directly coupled with antibody is used instead of using acridan and antibody co-labeled Amberlite microparticle. This acridan directly coupled with antibody is not immobilized. The complex formed would be chemiluminescent compound conjugated to a specific binding pair member—analyte-activator compound conjugated to another specific binding pair member instead of immobilized chemiluminescent compound conjugated to a specific binding pair member—analyte-activator compound conjugated to another specific binding pair member. Therefore, no solid phase support is required.

In one embodiment of microparticle immunoassay using labeled amberlite microparticles, the procedure and reagents are identical to the Example 15 of 20070264664 application except the adding of antioxidant/free radical scavenger. Take 100 ug/reaction of acridan and antibody co-labeled Amberlite microparticles of example 10 from 20070264664 application. The particles are washed three times with 1×PBS+0.05% Tween-20 and resuspended in sheep anti-mouse IgG F(ab¹)₂-HRP conjugate diluted 1:1.2×10⁶ in 1×PBS buffer containing 1% BSA and 1% sucrose. The particle suspension is dispensed into 26 wells of a white polystyrene 96 well plate. IgG standards in sheep anti-mouse IgG F(ab¹)₂-HRP conjugate solution are prepared by 2-fold serial dilution to result in final concentrations of 100 ng/mL-0.048 ng/mL or 0 ng/mL in the wells. The respective standards and zero are dispensed into wells to make a final reaction volume of 50 uL/well. The plate is incubated for 1 hr at room temperature on a plate shaker. Next optimal amount of antioxidant/free radical scavenger is added. The plate is transferred to a plate luminometer. Without removing the conjugate solution, luminescence is generated by sequentially injecting 100 uL of trigger solution, and reading the integrated intensity in each well for 5 seconds. A plot of the resulting assay allows quantitation of IgG. The optimal amount of antioxidant/free radical scavenger can be determined experimentally using the above procedure, for example, if VC is used, 2-fold serial dilution to result in final concentrations of 500 ng/mL-0.048 ng/mL or 0 ng/mL in the wells of VC is added to the wells containing either no analyte or 50 ng/ml analyte and the VC concentration give the best signal/noise ratio is selected as optimal amount.

In another embodiment of microplate immunoassay using unlabeled capture antibody and labeled BSA, the procedure and reagents are similar to the Example 13 of 20070264664 application except the adding of antioxidant/free radical scavenger or SOD and luminol is used instead of acridan. A 50 uL aliquot of unlabeled sheep anti-mouse IgG (H+L) 40 ug/mL of 1×PBS is added to coat each of 26 wells of a white polystyrene 96 well plate. The plate is agitated for 5 minutes at room temperature on an orbital shaker. The solution is removed and the wells washed three times with 1×.PBS+0.05% Tween-20, removing all wash buffer after each step. The luminol labeled BSA (10%) is diluted in 50 uL/mL in PBS buffer+1% sucrose. A 100 uL aliquot is added to each of 26 wells of a white polystyrene 96 well plate. The plate is held for 1 hr at 37 degree. The solution is removed and the wells washed three times with PBS+0.05% Tween-20, removing all wash buffer after each step. Sheep anti-mouse IgG F(ab¹)₂-HRP conjugate is diluted 1:1.2.times.10⁶ in a conjugate buffer comprising 1% BSA and 1% sucrose in 1×.PBS. Aliquots of diluted conjugate and suitable amount of antioxidant/free radical scavenger or SOD are dispensed into the 26 wells. IgG standards containing from 100 ng/mL-0.048 ng/mL were prepared by 2-fold dilution along with a 0 ng/mL solution in anti-IgG F(ab¹)₂-HRP conjugate solution. The standards and zero are dispensed into wells achieving a final volume 50 uL/well. The plate is incubated 1 hr at room temperature on the plate shaker. The plate is transferred to a plate luminometer. Without removing the conjugate solution, luminescence is generated by sequentially injecting 100 uL of trigger solution (the trigger solution use 1 uM p-iodophenol instead of the p-hydroxy-cinnamic acid), and reading the integrated intensity in each well for 5 seconds. Certain p-nitrophenol derivatives such as p-nitrophenol, o-methoxyphenol, p-methoxyphenol and 4-hydroxy-3-methoxycinnamic acids can also be antioxidant/free radical scavenger for this kind of assay.

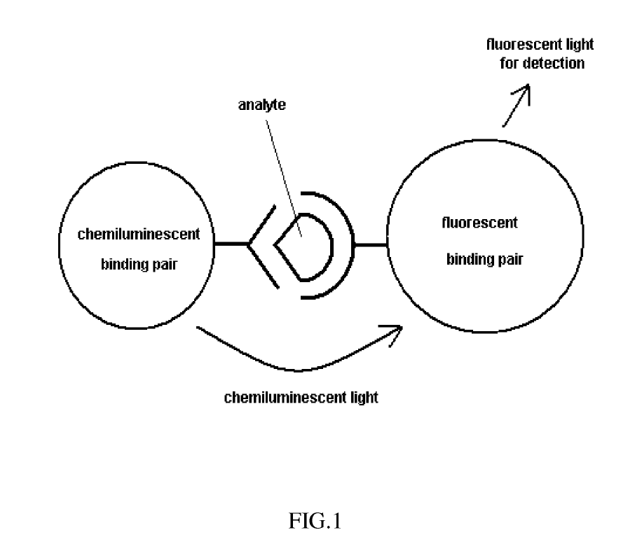

The current invention also discloses new methods to detect analyte using a chemiluminescent (bioluminescent) binding pair-fluorescent binding pair system in none separation or homogenous assay format. The methods can be used in both single analyte detection as well as multiple analyte detection (multiplex test). They can also be used in flowcytometer type assay. The general principle is described in FIG. 1.

The chemiluminescent binding pair has affinity group(s) that can bind with part of the analyte while the fluorescent binding pair has affinity group(s) that can bind with other part of the analyte. After mix with the sample, the chemiluminescent binding pair will form a complex with the analyte and the fluorescent binding pair. Because the chemiluminescent binding pair is very close to the fluorescent binding pair after binding. The light generated from chemiluminescent binding pair will be absorbed by fluorescent binding pair and the fluorescent binding pair will emit fluorescent light. The intensity of light emitted by the fluorescent binding pair is proportional to the amount of the binding complex formed and therefore it can be used to indicate the amount of the analyte in the sample.

The affinity group or groups can be any chemical or biological functionality with affinity for certain analytes. They include, but are not limited to, DNA, PNA (peptide nucleic acid), polynucleotides, antibody, antigen, aptamers, chelator, metals, lipophilic molecules, hydrophilic molecules, ionic molecules (such as acidic and basic molecules), dendrimer, polymers having affinity groups and other structures having specific affinity interactions with certain analytes. Preferably affinity group(s) is selected from antibody and aptamers. They can also be nucleic acid if the target analyte is nucleic acid. In addition to antibody-based and nucleic acid-based systems, other specific binding pairs as are generally known to one of ordinary skill in the art of binding assays can serve as the basis for test methods according to the present invention. The fluorescein/anti-fluorescein, digoxigenin/anti-digoxigenin, and nitrophenyl/anti-nitrophenyl pairs are exemplary.

The chemiluminescent binding pair also has chemiluminescent moiety that can generate chemiluminescent light under suitable condition. The chemiluminescent moiety can be chemiluminescent group(s) or chemiluminescent molecule(s) or chemiluminescent particle. The chemiluminescent binding pair can be in the form of solid phase; e.g, both chemiluminescent moiety and affinity group(s) are immobilized on solid support. The chemiluminescent binding pair can also be chemiluminescent moiety and affinity group(s) directly coupled together or conjugated together though a linker. The fluorescent binding pair also has fluorescent moiety that can generate fluorescent light under suitable condition. The fluorescent moiety can be fluorescent group(s) or fluorescent molecule(s) or fluorescent particle. The fluorescent binding pair can be in the form of solid phase; e.g, both fluorescent moiety and affinity group(s) are immobilized on solid support. The fluorescent binding pair can also be fluorescent moiety and affinity group(s) directly coupled together or conjugated together though a linker or carrier.

Chemiluminescent or fluorescent moiety may be coupled to the carrier either permanently (non-releasable) or through a cleavable (releasable) bond, e.g., photo-labile bond, chemical—labile bond such as an acid sensitive bond or a detachable bond, e.g., polynucleotide base pairing. The affinity group may be indirectly coupled to the carrier through a linker or an adaptor through, for example, a ligand-receptor binding (e.g., biotin-avidin) or hybridization between a polynucleotide and its complementary sequence. The carrier entity can be a polymer, a microparticle, or a combination of the two. Appropriate natural or synthetic polymers include, but are not limited to, oligomers (such as peptides), linear or cross-linked polymers (such as poly lysine, poly acrylic acid, proteins) or highly branched macromolecules (such as dendrimers). A chemical, biological or physical entity can be used as a carrier as long as it has multiple functional groups that allow direct or indirect conjugation of multiple numbers of signal (chemiluminescent/fluorescent) compounds/groups and affinity groups. The more functional groups a carrier has, the better amplification it will provide. One of the preferred carriers is a microparticle because it can be coated with a large number of functional groups such as carboxyl group or primary amine. Preferred size of microparticles is in the range of nanometer to micrometer in diameter. Suitable microparticles include, but are not limited to, microspheres, nanoparticles, liposomes, microcapsules and etc. In some embodiments, preferred size of particle is between 5 nanometers (nm) to 100 micrometers (μm) in diameter. Many vendors (e.g. Bangslabs Inc, Spherotech, Inc. Seradyn, Inc.) provide particles suitable for current invention.

Solid supports useful in the practice of the present invention can be of various materials, shapes, and sizes. Materials already in use in binding assays including microwell plates of the 96-well, 384-well or higher number varieties, test tubes, sample cups, plastic spheres, cellulose, paper or plastic test strips, latex particles, polymer particles, silica particles, magnetic particles, especially those having average diameters of 5 nm-100 um, and nanoparticles of various materials can all provide a useful solid support for attachment of chemiluminescent/fluorescent groups and for immobilizing specific affinity groups. Preferably, the microparticle is polymer based such as varieties of micro spheres. The preferred make of microspheres is polystyrene or latex material. However, any type of polymeric make of micro spheres is acceptable including but not limited to brominated polystyrene, polyacrylic acid, polyacrylonitrile, polyacrylamide, polyacrolein, polybutadiene, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethyhnethacrylate, or combinations thereof. The polymeric bead can be made easily by polymerization of monomers such as varieties of acrylates, styrenes, diene compounds or their derivatives. Suitable microspheres and the making of them are also available from many patents such as U.S. Pat. No. 6,649,414 and the reference they cited. Many vendors (such as Cortex biochem Inc, CA; Seradyn, Inc. IN; Dynal Biotech Inc., NY; Spherotech, Inc. IL; Bangs Laboratories, Inc. IN; Polysciences, Inc. PA) also provide suitable microspheres and micro particles and provide customer manufacture service. The microspheres can be either non cross linked or cross linked (such as contain 0.1 to 30% of a cross-linking agent, e.g. divinyl benzene, ethylene glycol dimethacrylate, trimethylol propane trimethacrylate, or N,N′ methylene-bis-acrylamide or other functionally equivalent agents known in the art). Preferably the microparticle is uniform in size and shape and preferably each microparticle contains the same or similar amount of signal groups/compounds for high sensitive detection of certain analyte. The microparticle may have additional surface functional groups that include, but are not limited to, carboxylates, esters, alcohols, carbamides, aldehydes, amines, sulfur oxides, nitrogen oxides, or halides. The functionality of the microparticles' surface groups gives the microparticle their coupling capability allowing chemical binding of analytical reactants. In addition to functional groups on microparticles the signal molecules such as dyes themselves can also carry chemically reactive functional groups, which in addition to groups listed above, can also be carboxylic acid, carboxylic acid succinimidyl ester, carboxylic acid anhydride, sulfonyl chloride, sulfonyl fluoride, hydrazine derivatives, acyl azide, isocyanate, haloacetamide, phenols, thiols, and ketones. These functional groups are useful for attachment of analytical reactants, i.e., classical, commonly used reactants such as antibody, antigen (hapten), digoxigenin, or nucleic acid probe. These may also include reactants that can form specific, high-affinity conjugates such as avidin-biotin, receptor-ligand, ligand-ligate, enzyme-substrate, lectin-carbohydrate, protein A-immunoglobulin, etc.

A carrier may be first directly or indirectly coupled with signal group (such as chemiluminescent compounds or fluorescent compounds) or affinity groups or both and then directly or indirectly conjugated to yet another carrier of the same type or different type. For example, acridinium and an oligonucleotide probe can be first coupled to polylysine to generate an acridinium-polylysine-oligonucleotide probe complex, which is subsequently conjugated to a microparticle. This carrier-to-carrier coupling reaction can be repeated a number of times to achieve further amplification prior to, during, or after contacting the analyte. When microparticles or the like are used as carriers, signal groups such as chemiluminescent or fluorescent compounds may be encapsulated in the particles. Encapsulation may be performed through physical means, e.g., trapping, internal adsorption, or through chemical means, e.g., covalent coupling. Alternatively, signal groups such as chemiluminescent compounds can first be directly or indirectly coupled to a carrier (e.g., a polymer or nanoparticles) and then encapsulated in the particle. The chemiluminescent compounds encapsulated could be in the form of aggregate, e.g., small particles, powder, or crystals, which are preferably in nanometer size range. For example, when rare-earth element such as Eu is used, it could be in the form of Eu metal particles, Eu oxide particles or other Eu containing compounds aggregate. Suitable chemicals for encapsulating chemiluminescent or fluorescent compounds include, but are not limited to, polymers such as polystyrene and small organic compounds such as Si containing compounds can also be used to coat chemiluminescent or fluorescent compound particle to give the encapsulated SAS microparticles. Many vendors (such as Cortex biochem Inc, CA; Seradyn, Inc. IN; Dynal Biotech Inc., NY; Spherotech, Inc. IL; mBangs Laboratories, Inc. IN; Polysciences, Inc. PA) also provide encapsulated microspheres and encapsulation service. It is normally, but not always, preferred that large numbers of signal molecules, or their derivatives, are encapsulated in microparticles to increase the sensitivity. Signal molecule can be derivatized (such as attaching a lipophilic group to it) for high encapsulation rate.

Alternatively, the oxides of rare elements, e.g., Eu oxide, can also be directly coated with polymers with functional groups such as carboxyl group, which can be used for affinity group labeling. For example, 3-aminopropyltrimethoxysilane APTMS can be used to coat Eu₂O₃ nanoparticle or the like. The coated Eu₂O₃ particles or the like can be conjugated with affinity groups and used as for the assay. Rare earth elements or the like such as Eu or Tb or Sm may be encapsulated in microparticles in the form of ions through a chelator. The Eu chelate, e.g. EDTA-Eu, beta-diketone-Eu, TTA-Eu-TOPO, can be incorporated into the monomer covalently. Appropriate chelators include, but are not limited to, isothiocyanatobenzyl-EDTA or TTA derivatives with an acid group or EDTA-5-aminosalycilic acid or 4,7-bis(chlorosulfophenyl)-1,10-phenanthroline-2,9-dicarboxylic acid (BCPDA). These chelators, or EU chelates, can be coupled to amine containing monomers such as 4-amino styrene through an amide bond linkage. The resultant Eu chelate containing monomer can be subsequently polymerized into microparticle or copolymerized with other monomer to give desired microparticle. Alternatively, the chelator, e.g., diketone (preferably 1,3-diketones such as acetylacetonate, benzoylacetonate, benzoylbenzoate, trifluoro-2-furylacetylacetone acetylacetonate, benzoylacetonate, benzoylbenzoate, trifluoro-2-furylacetylacetone), can be first incorporated into the monomer, which is polymerized or copolymerized with other monomer to form desired microparticles. The microparticles are then swelled or perforated and then incubated with Eu salt solution to allow Eu incorporation via the chelator molecules. Currently there is commercial available rare earth element containing microparticles available.

Similarly, chemiluminescent or fluorescent compound (or their derivatives) having reactive groups (such as amine group or carboxyl group) can also be coupled to monomers containing reactive group (such as 4-amino styrene) and then polymerized or copolymerized to give encapsulated micro sphere. Alternatively, the microsphere can be made to have reactive group (such as amine group) inside (such as those generated from 4-amino styrene) and then couple chemiluminescent or fluorescent compound (or their derivatives, such as acridinium NHS ester) with reactive groups (such as carboxyl group) to the micro sphere. The resulting microspheres will have chemiluminescent or fluorescent compound covalently encapsulated inside. The surface of the resulting microspheres can be modified for with affinity groups coupling. Cross-linked microspheres are more convenient for encapsulation and internal coupling since they normally cannot be dissolved in organic solvent. If non-cross linked micro sphere is used, the solvents during process need to be carefully examined to avoid dissolution. More examples of structure that can be used as chemiluminescent or fluorescent binding pairs can be found in US patent application 20050191687.

Appropriate CL by the chemiluminescent moiety here includes, but is not limited to, both direct chemiluminescence such as that generated with enzymes or acridinium and electro chemiluminescence such as that generated with rare earth elements. Chemiluminescent molecule(s)/compound(s) can be anything that generates light signal under appropriate conditions or the precursors that gives rise to such compounds. Examples for this type of compounds include both chemiluminescent compound (e.g., acridinium, proteins that can generate light, enzymes that can catalyze CL reaction) and electrochemolumincescent agents (e.g., certain organic compounds or metal elements in appropriate chelators).

Appropriate fluorescent groups here includes, but is not limited to, anything that generates fluorescent light signal under appropriate conditions or the precursors or derivatives that gives rise to such compounds. Examples for this type of compounds include both fluorescent compounds, proteins that can generate fluorescent light. A non limiting list is given here: fluorescent squaraine dyes, e.g., red dye which is 1,3-bis [(1,3-dihydro-1,3,3-rimethyl-2H-indol-2-ylidene)methyl]-2,4-dihydroxy-cycl obutenediylium, bis(inner salt); orange dye, e.g. 2-(3,5-dimethylpyrrol-2-yl)-4-(3,5-dimethyl-2H-pyrrol-2-ylidene)-3-hydroxy-2-cyclobuten-1-one; cyclobutenedione derivatives, substituted cephalosporin compounds, fluorinated squaraine compositions, symmetrical and unsymmetrical squaraines, alkylalkoxy squaraines, or squarylium compounds; phthalocyanines and naphthalocyanines; 3-Hydroxypyrene 5,8,10-Tri Sulfonic acid, 5-Hydroxy Tryptamine, 5-Hydroxy Tryptamine (5-HT), Acid Fuhsin, Acridine Orange, Acridine Red, Acridine Yellow, Acriflavin, AFA (Acriflavin Feulgen SITSA), Alizarin Complexon, Alizarin Red, Allophycocyanin, ACMA, Aminoactinomycin D, Aminocoumarin, Anthroyl Stearate, Aryl- or Heteroaryl-substituted Polyolefin, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, BOBO 1, Blancophor FFG Solution, Blancophor SV, Bodipy Fl, BOPRO 1, Brilliant Sulphoflavin FF, Calcien Blue, Calcium Green, Calcofluor RW Solution, Calcofluor White, Calcophor White ABT Solution, Calcophor White Standard Solution, Carbocyanine, Carbostyryl, Cascade Blue, Cascade Yellow, Catecholamine, Chinacrine, Coriphosphine O, Coumarin, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH-CH3, DAPI, Diamino Phenyl Oxydiazole (DAO), Dimethylamino-5-Sulphonic acid, Dipyrrometheneboron Difluoride, Diphenyl Brilliant Flavine 7GFF, Dopamine, Eosin, Erythrosin ITC, Ethidium Bromide, Euchrysin, FIF (Formaldehyde Induced Fluorescence), Flazo Orange, Fluo 3, Fluorescamine, Fura-2, Genacryl Brilliant Red B, Genacryl Brilliant Yellow 10OGF, Genacryl Pink 3G, Genacryl Yellow SGF, Gloxalic Acid, Granular Blue, Haematoporphyrin, Hoechst 33258 (bound to DNA), Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nile Red, Nitrobenzoxadidole, Noradrenaline, Nuclear Fast Red, Nuclear Yellow, Nylosan Brilliant Flavin EBG, Oregon Green, Oxazine, Oxazole, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Propidium Iodide, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Rose Bengal, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulphO Rhodamine B Can C, Sulpho Rhodamine G Extra, Tetracycline, Texas Red, Thiazine Red R, Thioflavin S, Thioflavin TCN, Thioflavin 5, Thiolyte, Thiozol Orange, Tinopol CBS, TOTO 1, TOTO 3, True Blue, Ultralite, Uranine B, Uvitex SFC, Xylene Orange, XRITC, YO PRO 1, or combinations thereof; and the derivatives of them. The lists of suitable fluorescent compounds/groups are also available from many patents such as U.S. Pat. No. 6,649,414 and the reference they cited. Derivatives of known fluorescent compounds (such as being attached with a reactive groups, e.g. an amine group or an carboxyl group) can also be used as long as they still have fluorescent property. J-aggregated dye or J-aggregated dye containing particles can also be used in the fluorescent binding pair, e.g. those described in many patents such as US patent application 20090047688 and the references it cited. If micro/nano particle are used in the fluorescent binding pair, fluorescent compounds/groups can be either coated on the surface of the particle or embedded inside the particle or both.

In some embodiments the preferred chemiluminescent compounds are capable of being oxidized to produce CL in the presence of the activator and/or a trigger solution. An exemplary class of compounds which by incorporation of a linker and reactive group could serve as the chemiluminescent label include luminol, and structurally related cyclic hydrazides including isoluminol, aminobutylethylisoluminol (ABEI), aminohexylethylisoluminol (AHEI), 7-dimethylaminonaphthalene-1,2-dicarboxylic acid hydrazide, ring-substituted aminophthalhydrazides, anthracene-2,3-dicarboxylic acid hydrazides, phenathrene-1,2-dicarboxylic acid hydrazides, pyrenedicarboxylic acid hydrazides, 5-hydroxy-phthalhydrazide, 6-hydroxyphthalhydrazide, as well as other phthalazinedione analogs disclosed in U.S. Pat. No. 5,420,275 and in U.S. Pat. No. 5,324,835. Another class of chemiluminescent moieties include acridinium/acridan esters, thioesters and sulfonamides disclosed in U.S. Pat. Nos. 5,491,072 and 6,030,803. Another class of chemiluminescent moieties includes the heterocyclic compounds disclosed in U.S. Pat. Nos. 5,922,558 and 6,891,057 as well as US patent application 20070264664. It is considered that any compound known to produce CL by the action of hydrogen peroxide and a peroxidase will function as the chemiluminescent moiety used in the present invention. Numerous such compounds of various structural classes, including xanthene dyes, aromatic amines and heterocyclic amines are known in the art to produce CL under these conditions. Other examples of chemiluminescent compounds (e.g. dioxetane type molecules, luciferin) can be found from well-known patent, literatures and commercial venders. For example, lumigen provides many chemiluminescent compounds (e.g. Lumigen PS-atto, Lumigen PS-2, Lumigen PS-3, Lumigen TMA-6, Lumigen TMA-3 can be used to emit light when mixed with peroxidase).

The activator is a compound/particle/solid phase that effects the activation of the chemiluminescent compound so that, in the presence of the trigger solution (sometimes no trigger is required), CL is produced. Compounds capable of serving as the activator include transition metal salts and complexes and enzymes, especially transition metal-containing enzymes, most especially peroxidase enzymes. Transition metals useful in activator compounds include those of groups 3-12 of the periodic table, especially iron, copper, cobalt, zinc, manganese, and chromium. Gold nanoparticles can also be used as activators. It should be noted that the activator molecules responsible for signal generation may sometimes operate within a physically confined radius and only have contact with a finite supply of chemiluminescent compound. This would seem to preclude large catalytic turnover in cases where the activator possesses that potential. The peroxidase which can undergo the chemiluminescent reaction include lactoperoxidase, microperoxidase, myeloperoxidase, haloperoxidase, e.g. vanadium bromoperoxidase, horseradish peroxidase, fungal peroxidases such as lignin peroxidase and peroxidase from Arthromyces ramosus and Mn-dependent peroxidase produced in white rot fungi, and soybean peroxidase. Other peroxidase mimetic compounds which are not enzymes but possess peroxidase-like activity including iron complexes, such as heme, and Mn-TPPS₄ are known which catalyze the chemiluminescent oxidation of substrates are explicitly considered to be within the scope of the meaning of peroxidase as used herein. Conjugates or complexes of a peroxidase and a biological molecule can also be used in the method for producing CL, the only proviso being that the conjugate display peroxidase activity. Biological molecules which can be conjugated to one or more molecules of a peroxidase include DNA, RNA, oligonucleotides, antibodies, antibody fragments, antibody-DNA chimeras, antigens, haptens, proteins, lectins, avidin, streptavidin and biotin. Complexes including or incorporating a peroxidase, such as liposomes, micelles, vesicles and polymers which are functionalized for attachment to biological molecules, can also be used in the methods of the present invention. Other example of activator can be found in US patent application 20070264664. The trigger solution provides a reactant necessary for generating the excited state compound necessary for CL. The reactant may be one necessary for performing the chemiluminescent reaction by reacting directly with the chemiluminescent label. It may serve instead of or in addition to this function to facilitate the action of the activator compound. This will be the case, for example, when the activator is a peroxidase enzyme. In a preferred embodiment the trigger solution comprises a peroxide compound. The peroxide component is any peroxide or alkyl hydroperoxide capable of reacting with the peroxidase. Preferred peroxides include hydrogen peroxide, urea peroxide, and perborate salts. Incorporation of certain enhancer compounds into the trigger solution promotes the CL of the enzyme. Included among these enhancers are phenolic compounds and aromatic amines known to enhance other peroxidase reactions. Preferred enhancers include but are not limited to: p-phenylphenol, p-iodophenol, p-bromophenol, p-hydroxycinnamic acid, p-imidazolylphenol, acetaminophen, 2,4-dichlorophenol, 2-naphthol and 6-bromo-2-naphthol. Mixtures of more than one enhancer from those classes mentioned above can also be employed. The trigger solution can also contain one or more detergents or polymeric surfactants to enhance the luminescence efficiency of the light-producing reaction or improve the signal/noise ratio of the assay. Nonionic surfactants useful in the practice of the present invention include by way of example polyoxyethylenated alkylphenols, polyoxyethylenated alcohols, polyoxyethylenated ethers and polyoxyethylenated sorbitol esters. Monomeric cationic surfactants, including quaternary ammonium salt compounds such as CTAB and quaternary phosphonium salt compounds can be used. Polymeric cationic surfactants including those comprising quaternary ammonium and phosphonium salt groups can also be used for this purpose. More examples of trigger can be found in US patent application 20070264664.

When select the chemiluminescent moiety and fluorescent moiety pair for the assay, the spectrum of chemiluminescent light needs to overlap with the excitation spectrum of the fluorescence (FL) of the fluorescent moiety. Light emitted by the present method can be detected by any suitable known means such as a luminometer, x-ray film, high speed photographic film, a CCD camera, a scintillation counter, a chemical actinometer or visually. Each detection means has a different spectral sensitivity. The human eye is optimally sensitive to green light, CCD cameras display maximum sensitivity to red light, X-ray films with maximum response to either UV to blue light or green light are available. Choice of the detection device will be governed by the application and considerations of cost, convenience, and whether creation of a permanent record is required. In those embodiments where the time course of light emission is rapid, it is advantageous to perform the triggering reaction to produce the CL in the presence of the detection device. As an example the detection reaction may be performed in a test tube or microwell plate housed in a luminometer or placed in front of a CCD camera in a housing adapted to receive test tubes or microwell plates.

In some embodiments, the chemiluminescent compound described above is used to form the chemiluminescent binding pair. Examples of preferred chemiluminescent compounds include chemiluminescent acridan, acridinium, ABEI, luminol, isoluminol and dioxetane type compounds. Examples of these chemiluminescent binding pair include these chemiluminescent compounds labeled antibody, antigen, nucleic acid or aptamer such as acridan labeled antibody, acridinium labeled antibody, luminol labeled nucleic acid and etc. They can also be in the form of chemiluminescent compounds and affinity groups immobilized on solid form as described above such as acridinium and antibody co immobilized on microsphere and acridan and antibody co immobilized on micro well plate as described in 20070264664 application. The activator and/or trigger that can initiate the CL can be in the reaction solution. For example, H₂O₂ other peroxide and NaOH for acridinium, peroxidase and H₂O₂ or other peroxide for luminol and acridan. The activator and/or trigger can be added at the beginning of the assay or right before the detection step after the binding is performed.

In one embodiment, the acridan-labeled antibody and acridan and antibody co-labeled microparticles is made according to the procedure described in the 20070264664 application. Take 100 ug/reaction of acridan and antibody co-labeled Amberlite microparticles of example 10 from 20070264664 application. The particles are washed three times with 1×PBS+0.05% Tween-20 and resuspended in sheep anti-mouse IgG F(ab¹)₂-fluorescein conjugate diluted 1:1.2×10⁶ in 1×PBS buffer containing 1% BSA and 1% sucrose. The particle suspension is dispensed into 26 wells of a white polystyrene 96 well plate. IgG standards in sheep anti-mouse IgG F(ab¹)₂-fluorescein conjugate solution are prepared by 2-fold serial dilution to result in final concentrations of 1000 ng/mL-0.048 ng/mL or 0 ng/mL in the wells. The respective standards and zero are dispensed into wells to make a final reaction volume of 50 uL/well. The plate is incubated for 1 hr at room temperature on a plate shaker. The plate is transferred to a luminometer. Without removing the conjugate solution, luminescence is generated by sequentially injecting 100 uL of trigger solution (Trigger solution (10 uL) containing 25 mM tris, pH 8.0, 8 mM p-hydroxycinnamic acid, 1 mM EDTA, 0.2% Tween-20 and 0.1 M urea peroxide) and 100 ul 0.05M horseradish peroxidase in 0.05M PBS buffer, and reading the RLU using at the wavelength at 510-530 nm using a filter. The reading at 510-530 nm is the fluorescence (FL) which indicates the concentration of the analyte IgG. Alternatively, micro plate can be used as solid support. The procedure and reagents can be made according to the Example 13 of 20070264664 application. A 50 uL aliquot of unlabeled sheep anti-mouse IgG (H+L) 40 ug/mL of 1×PBS is added to coat each of 26 wells of a white polystyrene 96 well plate. The plate is agitated for 5 minutes at room temperature on an orbital shaker. The solution is removed and the wells washed three times with 1×.PBS+0.05% Tween-20, removing all wash buffer after each step. Acridan labeled BSA in example 7 of 20070264664 application is diluted in 50 uL/mL in PBS buffer+1% sucrose. The plate is held for 1 hr at 37 degree. The solution is removed and the wells washed three times with PBS+0.05% Tween-20, removing all wash buffer after each step. Sheep anti-mouse IgG F(ab¹)₂-fluorescein conjugate is diluted 1:1.2.times.10⁶ in a conjugate buffer comprising 1% BSA and 1% sucrose in 1×.PBS. Aliquots of diluted conjugate are dispensed into the 26 wells. IgG standards in sheep anti-mouse IgG F(ab¹)₂-fluorescein conjugate solution are prepared by 2-fold serial dilution to result in final concentrations of 1000 ng/mL-0.048 ng/mL or 0 ng/mL in the wells. The respective standards and zero are dispensed into wells to make a final reaction volume of 50 uL/well. The plate is incubated for 1 hr at room temperature on a plate shaker. The plate is transferred to a luminometer. Without removing the conjugate solution, luminescence is generated by sequentially injecting 100 uL of trigger solution (Trigger solution (10 uL) containing 25 mM tris, pH 8.0, 8 mM p-hydroxycinnamic acid, 1 mM EDTA, 0.2% Tween-20 and 0.1 M urea peroxide) and 100 ul 0.05M horseradish peroxidase in 0.05M pH 8 PBS buffer, and reading the RLU using at the wavelength at 510-530 nm using a filter. The reading at 510-530 nm is the fluorescence (FL), which indicate the concentration of the analyte IgG. One can also determine the analyte concentration by the ratio of fluorescent emitting/chemiluminescent emitting (e.g. 520 nm/460 nm). The higher the ratio, the higher the concentration of the analyte. Acridan-antibody conjugate can be used directly instead of coating on the solid support, e.g 50 uL aliquot of Acridan-antibody conjugate at 40 ug/mL can be used instead.

The above embodiment use acridan as chemiluminescent moiety. Other chemiluminescent compounds can also be used instead (e.g. acridinium, ABEI, luminol, isoluminol and dioxetane). For example, luminol and antibody co-labeled Amberlite microparticles can used in the above example instead of using acridan and antibody co-labeled Amberlite microparticles. The trigger and/or activator and/or enhancer can to be adjusted for specific chemiluminescent compounds. For example, 5 uM p-iodophenol can be used in the trigger instead of 8 mM p-hydroxycinnamic acid. If acridinium is used, the activator should be NaOH and peroxide, the concentrations of which are well known to the skilled in the art (e.g. 0.1% H₂O₂, 0.05M NaOH). If 1,2-dioxetane AP substrates are used, the activator can be alkaline phosphatase instead of peroxidase and the enhancer can be TBQ.

The fluorescent binding pair is also not limited to anti-mouse IgG F(ab¹)₂-fluorescein conjugate. They can contain any fluorescent moiety that can emit FL upon excitation by the CL binding pair such as GFP, rhodamine 110. They can also in the form of solid support such as microparticles such as fluorescent particles and quantum dot, which immobilized with affinity groups for binding. Examples of suitable fluorescent particle include fluorescent-bioconjugated silica nanoparticles; Nanometer-sized fluorescent particles; bright and stable core-shell Fluorescent Silica Nanoparticles; Fluorescent dye-doped nanoparticles, fluorescent europium chelate-doped silica nanoparticles, Dye-Doped Silica Nanoparticles; dye-doped silica nanoparticles, Silica encapsulation of quantum dots, commerfcialy fluoresent microsphares/nanospheres (such as invitrogen FluoSpheres® carboxylate-modified microspheres, 0.04 μm, europium luminescent (365/610), FluoSpherescarboxylate-modified microspheres, 0.04 μm, platinum luminescent (390/650), FluoSpheres carboxylate-modified microspheres, 0.2 μm, platinum luminescent (390/650), TransFluoSpheres carboxylate-modified microspheres, (488/560), TransFluoSpheres carboxylate-modified microspheres (488/605), TransFluoSpheres carboxylate-modified microspheres, 0.04 μm (488/720), Streptavidin Coated Fluorescent Yellow Particles, Avidin Coated Fluorescent Yellow Particles, Biotin Coated Fluorescent Yellow Particles, Goat anti-Mouse IgG (Fc) Coated Fluorescent Yellow Particles from spherotech; Fluoro-Max streptavidin-coated flourescent particles from Seradyn); Functionalized Europium Oxide Nanoparticles; cadmium telluride nanoparticle, nanometer-size fluoresebt quantum dots (e.g. CdSe/Zns coreshell quantom dot655), QD655 and QD705; Luminescent quantum dots; Semiconductor fluorescent nanocrystals or quantum dots; Quantum Dot-Encoded Mesoporous Beads, luminescent quantum dots for multiplexed biological detection, multicolor quantum dots doping in mesoporous aaterials, Mesoporous silica beads embedded with semiconductor quantum dots and iron oxide nanocrystals, quantum dot-tagged fluorescent microbeads; Quantum-dot-tagged microbeads, fluorescent Au-nanodot.

It is known that high concentration of H₂O₂ or other peroxide may decrease the FL of some fluorescent compounds. Therefore, lower concentrations of H₂O₂ can be used in some applications (e.g. <1 mmol L-1, preferably <0.25 mM). However, many fluorescent microsphares/nanospheres having fluoresce compounds/quantum dot core/trapped inside and having an organic/inorganic coating/shell outside are resistant to high concentration of H₂O₂ or other peroxide therefore they can be used in the current invention. One can find many these kinds of microsphares/nanospheres from known art such as the reference listed above.

The selection of chemiluminescent moiety and fluorescent moiety pair need to meet the requirement describe above: when select the chemiluminescent moiety and fluorescent moiety pair for the assay, the spectrum of chemiluminescent light needs to overlap with the excitation spectrum of the FL of the fluorescent moiety. It is preferably that the maximal emitting wavelength of chemiluminescent light overlaps with the maximal excitation wavelength of FL of the fluorescent moiety. It is also preferred that the FL maximal emitting wavelength is far away from chemiluminescent maximal emitting wavelength, e.g. the difference is greater than 20 nm. It is known that the wavelength of CL from chemiluminescent compound can be adjusted by adjusting the structure of the chemiluminescent compound to meet the selection requirement (e.g. in U.S. Pat. No. 7,083,986—Near infrared chemiluminescent acridinium compounds and uses thereof, the wavelength of acridinium can be adjusted by modifying the acridinium structure). For example, most acridinium esters have maximal emission at 420-460 nm. Many dioxetane compounds have maximal emission at 450-480 nm. Many acridan compounds emit light at a maximum around 460 nm. Most adamantyl-stabilized 1,2-dioxetane substrates commercialized so far emit inherent CL in the range of 475-480 nm whileBZPD, a benzothiazole dioxetane phosphate, is colorless, nonfluorescent, and stable, and upon cleavage an emission maximum of 550 nm is observed. Luminol has maximal emission around 450 nm. For example, if luminol, acridan and acridinium having maximal emission around 430-460 nm is used as chemiluminescent moiety, the fluorescent moiety pair can be selected but not limited from fluorescein, Sypro Ruby, quantum dots (e.g. CdSe/Zns coreshell quantom dot655; QD705, Europium Oxide Nanoparticles; fluorescent microparticles (e.g. TransFluoSpheres carboxylate-modified microspheres, 0.04 μm (488/720), FluoSpherescarboxylate-modified microspheres, 0.04 μm, platinum luminescent (390/650), Fluorescent dye-doped nanoparticles. These fluorescent moiety can be coupled with IgG F(ab¹)₂ to form the fluorescent binding pair instead of the IgG F(ab¹)₂—fluorescein to be used in the examples above. The coupling procedure is well known to the skilled in the art and can be found in many publications. However if BZPD is used which has emission maximum of 550 nm, the preferred fluorescent moiety can be selected from Alexa™ 546, Calcium Orange™, Cyanine 3™, Nile Red, PBXL-1, TRITC, FluoSpheres® carboxylate-modified microspheres, 0.04 μm, red-orange fluorescent (565/580), FluoSpheres® carboxylate-modified microspheres, 0.5 μm, red fluorescent (580/605) because their fluorescent excitation wavelength is close to 550 nm and emitting wavelength is far away to 550 nm.

In another format, the activator is used as chemiluminescent moiety to couple with affinity groups to form the chemiluminescent binding pair and the chemiluminescent compounds for the activator is added in the assay mix to trigger the CL. For example, if the activator is peroxidase such as horseradish peroxidase, it can be coupled to an antibody or co immobilized on solid phase support with antibody to form the chemiluminescent binding pair. The assay mix can contain luminol, H2O2 and enhancer to trigger the CL. Example of activator are listed above as well as described in many reference such as those described in 20070264664 application. Examples of the chemiluminescent compounds include but not limited to acridan, acridinium, ABEI, luminol, isoluminol and dioxetane type compounds. In principle, any compounds/molecules/particles that can trigger the chemiluminescent can be used as activators. Some activators need to work with certain chemiluminescent compound for light emitting. For example, peroxidase and luminol is this kind of pair. Luciferase-luciferin is also this kind of pair. Therfore luciferase coupled with affinity groups can also be used as chemiluminescent binding pair either by directly coupling or via a linker or on a solid phase support. The reaction mix need to contain the luciferin and other molecules needed for the CL reaction. For example, both ATP and firefly luciferin is needed for the firefly luciferase type chemiluminescent binding pair. As described above, there are several types of luciferase and each of them needs their own type of luciferin. For example, firefly luciferase and click beetles luciferase need firefly luciferin or its analogue as well as ATP and Mg2+ for CL. Bacterial luciferase needs bacterial luciferin or its analogue, which is a reduced riboflavin phosphate (FMNH₂), which is oxidized in association with a long-chain aldehyde, oxygen. Cypridina luciferase needs cypridina luciferin or its analogue. Renilla luciferase use coelenterazine or its analogue. The activator can also be gold nanoparticles since it can trigger the chemiluminescent of luminol, isoluminol and acridan or the like the like at the presence of peroxide compound such as H₂O₂ or other molecules that can emit light at the presence of gold particle and peroxide.

Further more, many enzymes/molecules can convert the non-chemiluminescent substrate to chemiluminescent molecules which can emit light spontaneously or emit light under contain condition. These enzymes/molecules can also be used as activators to form chemiluminescent binding pair. The assay mix needs to contain the corresponding non-chemiluminescent substrate. For example, the invention described above illustrated many firefly luciferin derivative/conjugate molecules for certain enzyme detection. These enzymes convert these firefly luciferin derivative/conjugates to free firefly luciferin, which is a chemiluminescent molecule. Therefore these enzymes can be used to form the chemiluminescent binding pair. These enzymes are not limited to luciferin system. Any enzyme that can convert a non-chemiluminescent substrate to chemiluminescent molecules is suitable for the current invention. For example, acridan derivatives and 1,2-dioxetane derivatives can also be used as non-chemiluminescent substrate. Many vendors (e.g. lumigen) provide such acridan derivatives and dioxetane derivatives. For example, alkaline phosphatase can be use to trigger Lumigen APS-5 and Lumigen® PPD, Lumi-Phos® 530, Lumi-Phos® Plus to generate CL. Beta galactosidase can be used for Lumi-Gal 530 substrate. There are also many acridinium derivatives, acridan derivatives, 1,2-dioxetane derivatives, luciferin derivatives or the like can be used as substrate for certain enzyme to produce CL. Many of them are described in patents and literatures as well as the current invention. For example U.S. Pat. No. 6,586,196, US Patent Application 20070048812, 20070015790, 20060257863 and 20060073529 disclosed many chemiluminescent substrate for certain enzymes. These substrates or the like and the corresponding enzyme pair can be used in the current application described above. When 1,2-dioxetane derivatives type substrate is used, dioxetane enhancer such as onium groups (e.g. ammonium, cationic homopolymer or copolymer comprising positively charged onium groups, (vinylbenzyldimethylbenzylammonium chloride) (BDMQ), poly(vinylbenzyltrimethylammonium chloride) (TMQ), poly(vinylbenzyltributylammonium chloride) (TBQ), poly(vinylbenzyltri(n-pentyl)ammonium chloride) (TPQ), poly(vinylbenzyltributylphosphonium chloride) (TB), poly(vinylbenzyltrioctylphosphonium chloride) (TO) can be coated to the microparticle/nanoparticles of the fluorescent binding pair to enhance the CL of dioxetane.

One of the formats is to use peroxidase as chemiluminescent binding pair and the chemiluminescent molecule that can be triggered by peroxidase (e.g. luminol, isoluminol, acridan and etc. and if necessary, their enhancer) is in the assay buffer. The chemiluminescent molecule can be added at the beginning or only at the detection stage. One embodiment using peroxidase is described in example 1. The peroxidase and affinity groups can also be coated on microparticles or nano particles as described above. The fluorescent binding pair can also utilize other fluorescent moiety instead of QD. For example, it can be selected from the fluorescent molecule/particles described above such as Eu oxide particle, Fluorescent Silica Nanoparticles, Nanometer-sized fluorescent particles, Core-Shell Fluorescent Silica Nanoparticles, Fluorescent dye-doped nanoparticles, fluorescent europium chelate-doped silica nanoparticles, Silica encapsulation of quantum dots, commercially fluorescent microspheres/nanospheres, Quantum Dot-Encoded Mesoporous Beads, quantum dot-tagged fluorescent microbeads and etc. The conjugation of affinity group with these fluorescent groups is well known to the skilled in the art and the protocol can be readily adopted from known procedures described in publications. In some embodiments, it is preferred that larger microparticle (e.g. >100 nm, preferably >500 nm) be used because they have bigger chemiluminescent light absorbing surface and contain more fluorescent moiety therefore can emit stronger fluorescence. If the chemiluminescence (CL) binding pair is also in particle form, using smaller size particle (compared with the fluorescent binding pair) can reduce the background light emitting in some cases. Because gold nanoparticles (e.g. 6-99 nm GNPs) can also function like peroxidase, it can be used in this kind of assay instead by coupling with affinity groups to form chemiluminescent binding pair.

Another format is to use luciferase to make the chemiluminescent binding pair. An example using quantum dot as fluorescent binding pair is given below. The assay is to detect the peptide human chronic gonadotropin (hCG) in the serum sample. The first monoclonal antibody (1^{st} affinity group) for human chronic gonadotropin (hCG) is coated on QD705, the second monoclonal antibody (2^{nd} affinity group) for hCG, which recognizes a different portion of the hCG molecule than that recognized by the first monoclonal antibody is coupled with firefly luciferase. One embodiment using luciferase is described in example 2. The fluorescent binding pair can also utilize other fluorescent moiety instead of QD. For example, it can be selected from the fluorescent molecule/particles described above such as Eu oxide particle, Fluorescent Silica Nanoparticles, Nanometer-sized fluorescent particles, Core-Shell Fluorescent Silica Nanoparticles, Fluorescent dye-doped nanoparticles, fluorescent europium chelate-doped silica nanoparticles, Silica encapsulation of quantum dots, commercially fluorescent microspheres/nanospheres, Quantum Dot-Encoded Mesoporous Beads, quantum dot-tagged fluorescent microbeads and etc. For example, when Firefly Luciferase-antibody conjugate 2 μg/mL is used in the above assay in example 2, FluoSpheres® carboxylate-modified microspheres, 0.2 μm, red fluorescent (580/605) coated with antibody 20 μg/mL can be used instead and the detection is selected for >600 nm because firefly luciferase emit maximal at 560 nm. In another example, if 5 ug/ml renilla luciferase-antibody conjugate is used in the above assay, 25 ug/ml Eu₂O₃ nanoparticle-antibody conjugate (610 nm fluorescence emission with excitation at 460 nm) can be used instead the detection is set for >600 nm. The conjugation of affinity group with these chemiluminescent/fluorescent groups is well known to the skilled in the art and the protocol can be readily adopted from known procedures described in publications. In some embodiments, it is preferred to larger microparticle (e.g. >100 nm, preferably >500 nm) because they have bigger chemiluminescent light absorbing surface and can emit stronger fluorescence.

In the assay described above, all the chemiluminescent binding pair will emit light no matter it is bind with analyte or not. All fluorescent bind pair will emit fluorescent light no matter they bind with analyte all not if they adsorb the chemiluminescent light. In another word, non-bound chemiluminescent binding pair will also cause some non-bound fluorescent bind pair to emit fluorescent light, which will be detected. Since this light is not caused by analyte binding, they contribute to background noise. The current invention also provides a method to reduce this noise as well as chemiluminescent light and other background light, which may interfere the detection. The principle is to add certain compounds or particles that can specifically absorb the chemiluminescent light strongly but not or only weakly absorb the fluorescent light to the assay/detection mix. They should not emit light or only weakly emit light in the detection and major excitation wavelength of the fluorescent light after the absorption of the chemiluminescent light. Examples of them include dye, pigment or quencher. It can be done by adding one species of dye/pigment/quencher (e.g. gold nanoparticles quencher) or the combination of several species.

It is preferred that the selected compounds/particles will have high absorption coefficient (absorptivity) in the major excitation wavelength region of the chemiluminescent binding pair for the fluorescent binding pair and low absorption coefficient in the major fluorescent light emitting wavelength region or the detection wavelength region of the fluorescent light. Therefore the light from the non-bound chemiluminescent binding pair will be absorbed and not to excite the non-bound fluorescent bind pair to emit fluorescent light. Because the binding chemiluminescent binding pair and the binding fluorescent bind pair are very close to each other due to they form a sandwich structure with analyte, the light energy will be much easier to transfer from chemiluminescent binding pair to the fluorescent bind pair and also due to the local relatively low concentration of dye/pigment/quencher, the resulting fluorescence will not or only weakly affected. Because different dye/pigment/quencher has different absorption coefficient for different wavelength, the concentration (and combination if use multiple dye/pigment/quencher) of them in different application need to be adjusted accordingly to reach the best signal noise ratio. One can start from the concentration that almost completely absorb light in the target chemiluminescent light wavelength then decrease the concentration until it does not or only slightly decrease the fluorescence light for detection. For example, in the above example using renilla luciferase and QD 705, McCormick (aka Schilling) food coloring red or yellow dye can be added at the concentration of 0.005%˜0.0005%; QSY-35 quencher (abs max 470 nm) can be used. Other dye such as surf green dye (high abs in 470 nm) can also be used. When acridinium (Em 420 nm) is used, DABCYL acid quencher can be used since it has high abs at 425 nm. When firefly luciferase is used, QSY-9 quencher (abs max 562 nm) can be used.

This method can also be used in FRET (Fluorescence Resonance Energy Transfer) or the like. These assays utilize a distance-dependent excited state interaction in which emission of one fluorohore is coupled to the excitation of another. To reduce the background noise caused by the none-binding donor exciting none-binding acceptor, suitable amount of dye/pigment/quencher can be added. The dye/pigment/quencher or their combinations should have strong abs in the donor emitting wavelength utilized by the accepter but no or low abs in major accepter fluorescent light emitting wavelength region or the detection wavelength region of the accepter fluorescent light. They should not emit light or only weakly emit light in the detection and major excitation wavelength of the accepter fluorescent light. For example in the FRET assay described in BMC Biochem. 2002; 3: 27, one can add a dye having high abs at 490-550 nm but low abs at 580 nm to reduce the background therefore increase the sensitivity. In another example, if QD 650 is used as donor and Cy5 containing beads is used as acceptor, one can add a dye having high abs at 650 nm but low abs at 690 nm to reduce the background.

U.S. patent application Ser. No. 12/287,916 described a homogenous luciferase based assay utilizing the enzyme-channeling phenomenon. Because now engineered firefly luciferase can emit different wavelength of light than the native firefly luciferase, therefore this method can also be used for multiplex assay. For example, the luciferase-antibody conjugate use native firefly luciferase for analyte 1 and the luciferase-antibody conjugate for analyte 2 use engineered firefly luciferase that can emit a different wavelength of light. The two set of probes is mixed in one assay solution therefore the analyte 1 and 2 can be detected simultaneously beaded on the different wavelength of light emitting caused by their binding.

One variation of a method in the current invention is using the principle of the homogenous luciferase based assay described in U.S. patent application Ser. No. 12/287,916 utilizing the enzyme-channeling phenomenon. An enzyme channeling system is composed of two enzymes/molecules, in which the first enzyme/molecule acts on a substrate and produces a product that is the substrate of the second enzyme. Enzymatic action of the second enzyme produces a light signal, which will be used to excite the fluorescent binding pair to emit fluorescent light for detection. The second enzyme can be either coupled with the chemiluminescent binding pair or coupled with the fluorescent binding pair directly. In some embodiments, luciferase is coupled with affinity group (either directly or via a linker or solid support) as chemiluminescent binding pair; the luciferase substrate generating enzyme/molecule (e.g. firefly luciferin generating enzyme/molecule or ATP generating enzyme/molecule if firefly luciferase is used, coelenterazine generating enzyme/molecule if renilla luciferase is used, FMNH₂ producing enzyme/molecule if bacterial luciferase is used) is coupled to the fluorescent binding pair. When analyte is present, the luciferase and luciferase substrate generating enzyme/molecule will bind together and the luciferase substrate generating enzyme/molecule will provide substrate for luciferase and the luciferase will emit light, which will excite the fluorescent moiety in the bound complex to emit fluorescent light for detection. Yet another format is the luciferase substrate generating enzyme/molecule is coupled with affinity group (either directly or via a linker or solid support) as chemiluminescent binding pair (more precisely it function as a chemiluminescence activating binding pair) and the luciferase is coupled to the fluorescent binding pair. When analyte is present, the luciferase and luciferase substrate generating enzyme/molecule will bind together and the luciferase substrate generating enzyme/molecule will provide substrate for luciferase and the luciferase will emit light, which will excite the fluorescent moiety in the bound complex to emit fluorescent light for detection. Yet the third format is the luciferase substrate generating enzyme/molecule is coupled with affinity group (either directly or via a linker or solid support) as chemiluminescent binding pair and the luciferase is not coupled to the fluorescent binding pair, instead the luciferase is in free form in the assay mix or also coupled to the chemiluminescent binding pair and the fluorescent binding pair contains only the affinity group and fluorescent moiety.

It is need to know that in the current invention the term coupled with means either directly couple or couple via a linker or co immobilized on the solid support (e.g. nano/micro particle, microwell plate and etc) as described above.

In one embodiment described in example 3, the assay is to detect the peptide human chronic gonadotropin (hCG) in the serum sample. Another example is to use alkaline phosphatase—1^{st} antibody-Rhodamine Red conjugates 2 μg/mL (or alkaline phosphatase—1^{st} antibody-QD 705 conjugate 6 ug/ml) and Firefly Luciferase—2^{nd} antibody conjugate 2 μg/mL instead in the above assay described in example 3. Other assay condition is the same. However, a dye that has strong abs at 560 nm but low abs at 590 nm (or 705 nm for QD 705) can also be added to reduce the background light.

Yet the third example is to use 1^{st} antibody-QD 650 conjugate 2 μg/mL and alkaline phosphatase—2^{nd} antibody conjugate 2 μg/mL and free Firefly Luciferase 10 ug/ml instead in the above assay. Other assay condition is the same. Alkaline phosphatase—2^{nd} antibody-firefly luciferase conjugate 2 μg/mL can also be used instead of using free firefly Luciferase and alkaline phosphatase—2^{nd} antibody conjugate.

In the case of homogenous assay using firefly luciferase or the like that using firefly luciferin for CL described in U.S. patent application Ser. No. 12/287,916, another format is to use ATP generating enzyme coupled with affinity group (either in solid phase format or not) and firefly luciferin generating enzyme coupled with affinity group (either in solid phase format or not) to form an sandwich complex with the target analyte for detection. Firefly luciferase or the like utilizing ATP and firefly luciferin is added to the assay mix as well as substrate for ATP generating enzyme and substrate for firefly luciferin generating enzyme. No free luciferin and ATP need to be within the assay mix. When the ATP generating enzyme and firefly luciferin generating enzyme are close to each other by the bound analyte, the local ATP and luciferin concentration is high therefore trigger the local free luciferase to emit intensive light indicating the presence of analyte. Luciferin antibody and ATPS (ATP degradation enzyme) can also be added to eliminate the background light caused by none binding enzyme. The luciferase can also be co coupled with either ATP generating enzyme or firefly luciferin generating enzyme on the solid support or via a linker. Alternatively, luciferase can also be coupled with the 3nd affinity group which can form a triple binder complex with the analyte instead of the two binder sandwich complex. Fluorescent moiety that can be excited by the light of luciferase can also be coupled with ATP generating enzyme or firefly luciferin generating enzyme or both and in this case the detection would be for the fluorescence light. Fluorescent moiety can also be coupled to the luciferase as well. If renilla luciferase is used, coelenterazine generating enzyme can be coupled with first affinity group and renilla luciferase can be coupled with second affinity group to form an sandwich structure with analyte. At the presence of substrate of coelenterazine generating enzyme and no free coelenterazine added, they will emit light for detection. Similarly, fluorescent moiety that can be excited by the light of renilla luciferase can also be coupled with either renilla luciferase or coelenterazine generating enzyme (directly or via a linker or on solid phase) or both and the detection is for the fluorescence. If bacterial luciferase is used, FMNH₂ producing enzyme/molecule can be coupled with the first affinity group and the bacterial luciferase can be coupled with the second affinity group to produce light signal for analyte detection. If fluorescent moiety is also coupled with FMNH₂ producing enzyme/molecule—1^{st} affinity group or bacterial luciferase—2^{nd} affinity group or both, the fluorescence signal can be used for analyte detection. Another format using bacterial luciferase is FMNH2 producing enzyme/molecule can be can be coupled with the first affinity group and the NADH producing enzyme (which enable the activity of FMNH₂ producing enzyme) can be coupled with the second affinity group to produce light signal for analyte detection. The bacterial luciferase can be either in free format in the assay mix or coupled with one or both binding pair or coupled with the 3^{rd} affinity group to form the triple binder complex with the analyte. If fluorescent moiety is also coupled to one or two or all these 3 enzymes, the fluorescence signal can be used for analyte detection.

In general there are two scenarios in the luciferase type assay. One involves two enzymes: one luciferase substrate generating enzyme/molecule and the luciferase (e.g. ATP generating enzyme and firefly luciferase; coelenterazine generating enzyme and renilla luciferase). Another involves three enzymes/molecules: two types of luciferase substrate generating enzyme/molecule and the luciferase (e.g. ATP generating enzyme, luciferin generating enzyme and firefly luciferase; FMNH2 producing enzyme, NADH producing enzyme and bacterial luciferase). In the two-enzyme assay, the two enzymes are coupled with affinity groups and are brought close due to analyte binding to generate chemiluminescent light. The fluorescent moiety can be conjugated with either one of them or both if fluorescent light is for detection. In the three-enzyme assay, one enzyme can be placed in the assay mix and the other two can be coupled with affinity groups to bind with analyte forming two binding pair sandwich structure to generate chemiluminescent light for detection. Alternatively, three enzymes can couple with three affinity groups to form triple binding pair or two of the enzymes are co coupled with one affinity group (e.g. twp enzymes and affinity group are co immobilized on bead) and another enzyme is coupled with the 2^{nd} affinity group to bind with analyte for chemiluminescent detection. If fluorescent light needs to be used for detection, fluorescent moiety can be coupled with one or two or all of these three enzymes/binding pairs. The product of the enzyme should not be included in the assay mix if the enzyme is used. Some molecules have certain catalytic function therefore they can also be used as enzyme instead in the current invention (e.g. heme or gold nano particle can be used as peroxidase).

In some embodiments, luciferin immobilized on solid phase having affinity group or coupled with affinity group directly can be used as luciferin generating enzyme-affinity group conjugate instead. For example, U.S. Pat. No. 4,665,022 described an amino terminated firefly luciferin, which can covalently couple to a protein or other—COOH containing carrier retaining its bioluminescent properties as luciferase substrate. Firefly luciferin can also be absorbed to the solid phase support having positive charged surface (e.g. microparticles having amine surface group). In one application, amino terminated firefly luciferin is coupled with the first affinity group (or amino terminated firefly luciferin and the 1^{st} affinity group is co immobilized on a 0.1 um carboxylated particle), ATP generating enzyme is coupled with the 2^{nd} affinity group. No free luciferin is added to the assay mix while the substrate for the ATP generating enzyme and free luciferase is provided in the assay mix. At the presence of analyte, the formed binding complex will emit light for detection. ATP generating enzyme and the 2^{nd} affinity group can also be co immobilized on QD 650 and the detection will be for the fluorescence emission. In another format, only the 2^{nd} affinity group is immobilized on QD 650 and the assay mix contain free ATP and free luciferase, the detection is for the fluorescence emission of QD.

In some embodiments using fluorescent light for detection, the chemiluminescent dioxetane generating enzyme is coupled with affinity group to form the chemiluminescent binding pair, the fluorescent moiety (e.g. QD, fluorescent dye, fluorescent micro/nano particle, Eu oxide particle) is coupled with affinity group to form the fluorescent binding pair. In one embodiment described in example 4, the assay is to detect the peptide human chronic gonadotropin (hCG) in the serum sample.

Alternatively instead adding enhancer to the solution, dioxetane enhancer (e.g. ammonium, cationic homopolymer or copolymer comprising positively charged onium groups, BDMQ, Sapphire, Sapphire 11, TPQ, THQ, phosphonium polymers, and/or copolymers of ammonium and/or phosphonium monomers or the like) can be coated/immobilized on the Eu oxide particle surface. Other fluorescent microparticles previously described (e.g. Fluorescent dye-doped nanoparticles, Silica encapsulation of quantum dots, fluorescent europium chelate-doped silica nanoparticles, TransFluoSpheres carboxylate-modified microspheres (488/605)) as well as fluorescent dye can also be used instead of Eu oxide particles.

The current invention also discloses a chemiluminescent method for analyte detection. The chemiluminescent dioxetane-generating enzyme is coupled with affinity group (e.g. 1st antibody) to form the chemiluminescent binding pair, dioxetane enhancer moiety and affinity group (e.g. 2^{nd} antibody) is co immobilized on a non-fluorescent solid phase (e.g. non fluorescent micro/nano particle, microwell plate) as enhancing pair. The chemiluminescent binding pair, enhancing pair and analyte can form a sandwich structure complex upon binding. When the dioxetane substrate is added to the assay mix, the complex will emit strong enhanced chemiluminescent light for detection. The coating method of dioxetane enhancer can be readily adapted from the protocol described above by using non-fluorescent particle/surface instead. In one embodiemtn described in example 5, the assay is to detect the peptide human chronic gonadotropin (hCG) in the serum sample.

The methods described above all utilize a pair of binding pair, e.g. chemiluminescent binding pair-analyte-fluorescent binding pair. By directly couple the chemiluminescent moiety with the fluorescent moiety; a self-illuminating fluorescent structure is formed. For example, by couple the peroxidase with fluorescent dye or QD or micro/nano fluorescent particle or Eu oxide nano particle; the formed structure will emit fluorescent light at the presence of peroxide and acridan or luminol. By couple the alkaline phosphatase with fluorescent dye or QD or micro/nano fluorescent particle or Eu oxide nano particle; the formed structure will emit fluorescent light at the presence of 1,2-dioxetane AP substrate or other molecules that can emit light after been processed by AP. By couple ATP generating enzyme with QD or micro/nano fluorescent particle or Eu oxide nano particle, it will emit fluorescence light at the presence of luciferin and luciferase. By couple luciferin generating enzyme with QD or micro/nano particle or Eu oxide nano particle, it will emit fluorescence light at the presence of luciferase. Certain affinity group such as antibody can also be co immobilized on the particle to make it having affinity to certain analyte. Using different affinity group to pair with different fluorescent moiety (e.g. different QD that emit different fluorescence), they can be used for multiplex detection (e.g. to label the different markers in the tissue).

The above methods using chemiluminescent binding pair-analyte-fluorescent binding pair and reagents can also be used in multiplex detection (detect multiple analyte in one assay). For example, in order to detect two analyte molecules, two set of chemiluminescent binding pair-fluorescent binding pair are needed to form chemiluminescent binding pair 1-analyte1-fluorescent binding pair1 and chemiluminescent binding pair 2-analyte2-fluorescent binding pair2 to emit fluorescent signal for detection. Both sets of chemiluminescent binding pair-fluorescent binding pair should be present in the assay mix. The signal can be distinguished by using different fluorescent moiety in different fluorescent binding pair that emits different wavelength shape of fluorescent light. For example, fluorescent binding pair 1 uses QD 650 and fluorescent binding pair 2 uses QD 705 wile the chemiluminescent moiety can be the same or different as long as the condition for both chemiluminescent binding pairs emitting light is provided, therefore the fluorescent light in 650 nm will be used for detection of analyte 1 and the fluorescent light in 705 nm will be used for detection of analyte 2. The detection can be done simultaneously or sequentially. If it is done sequentially, e.g. detect the 650 nm first them detect the 705 nm, one can add a dye that have high abs for 650 nm but low abs for 705 nm before the detection for 705 nm. If different chemiluminescent moiety for different analyte is used and different chemiluminescent moiety requires different condition for CL, the detection can also be done sequentially by provide these conditions sequentially (e.g. add different reagent sequentially) to enable the chemiluminescent sequentially. In another format, the two chemiluminescence are initiated at the same time but the inhibitor for the CL 1 is added before the detection of analyte 2. The combination of providing conditional for CL 2 to initiate it and inhibit the CL 1 can be used too for detection of analyte 2.

There are many different types of fluorescent moiety such as fluorescent micro/nano particles and fluorescent dyes as well as fluorescent dye-doped nanoparticles, Silica encapsulation of quantum dots, quantum dot-tagged fluorescent microbeads can be used as long as they can emit different fluorescent light. By using more than two type of fluorescent moiety, more than two analyte can be detected therefore the multiplex test can be easily expanded to more analytes.

Another format for multiplex detection is to use the same fluorescent moiety but different chemiluminescent moiety for different analyte binding. These different chemiluminescent moiety need to require different condition for their CL or can be inhibited by different inhibitors/inhibiting condition for their CL. Therefore by providing these condition sequentially or adding inhibitor sequentially or both, one can select enable the CL of certain chemiluminescent moiety therefore selectively detect certain analyte. For example, if firefly luciferase-antibody for analyte 1 conjugate and renilla luciferase-antibody for analyte 2 conjugate are used as two chemiluminescent binding pair as well as QD 710-antibody for analyte 1 conjugate and QD 710-antibody for analyte 2 conjugate are used for the detection of analyte 1 and analyte 2, ATP and firefly luciferin is added in the assay mix for the detection of analyte 1 but no coelenterazine, after the detection of analyte 1, coelenterazine will be added to initiate the detection of analyte 2. Now the fluorescent light will be the sum of that generated from analyte 1 and analyte 2. Firefly luciferase CL inhibitor such as ATPs/firefly luciferin antibody/firefly luciferase antibody can be added to inhibit the fluorescence light interference from analyte 1.

These self-illuminating fluorescent micro/nano particle described in the current invention can also be used in flowcytometry type assay therefore eliminate the use of laser or reduce the number of laser need to be used. In current liquid array method, beads having different affinity groups are coded (such as using specific bead size, color and its intensity, different fluorescent dye or fluorescent dye combination, e.g. U.S. Pat. No. 6,514,295). During an assay, different beads bind with different analytes, which also bind to second affinity groups that have the same fluorescent tag. The resulting sandwich structure, coded beads-analyte-fluorescent tag, enables the identification of the beads and, consequently, the analyte or analytes present in the sample. Here the fluorescent tag serves as an indicator for the presence of analyte or analytes in the sample whereas the codes in the beads reveal which analyte or analytes are present in the sample. Each coded beads are coupled with a specific affinity group. The fluorescent color coded beads need to be illuminated by laser to generate fluorescent light.

When the self-illuminating fluorescent micro/nano particle is used, the chemiluminescent moiety (e.g. firefly luciferase, renilla luciferase, peroxidase, phosphatase, acridan, acridinum or other enzymes/molecules that can generate chemiluminescent light) can be permanently immobilized on the fluorescent micro/nano particle. When the corresponding substrate or reagent (e.g. luciferin for luciferase, luminol/acridan for peroxidase, NaOH/H₂O₂ for acridinium is mixed with assay solution or mixed at the detection cell/pathway right before the detection if the light is flash light type) is present, the chemiluminescent light will excite the fluorescent micro/nano particle similar to the laser. The ID code of the beads therefore can be determined. Therfore the need for this laser/s is not required. The bead's ID can also be determined by the combination of size and color. There are many ways to make fluorescent color coded microsphere such as those disclosed in above and in U.S. Pat. Nos. 6,649,414; 6,514,295 and 5,073,498 and the reference they cited. Many vendors (such as Cortex biochem Inc, CA; Seradyn, Inc. IN; Dynal Biotech Inc., NY; Spherotech, Inc. IL; Bangs Laboratories, Inc. IN; Polysciences, Inc. PA) also provide this kind of microspheres and microsphere manufacturing service. QD encoded fluorescent particle is also suitable for these kind of application. Example of flow cytometer can be used include those described in U.S. Pat. No. 5,032,381 and xMAP™ technology from Luminex or the like. In order to detect the analyte, affinity group for the analyte can be co immobilized on the particle and the detection for the analyte binding can be performed same as those in the liquid array technique (using fluorescent tag labelled second affinity group). A laser is needed to excite the fluorescent tag labelled second affinity group for the detection of the analyte. The fluorescent tag can also be excited by the chemiluminescent light on the beads therefore eliminate the need for laser.

Yet in another format, the detection is for the chemiluminescent binding pair-analyte-fluorescent particle sandwich structure. The coded fluorescent particle has affinity groups on its surface and the chemiluminescent moiety is not permanently immobilized on the particle. The binding of the chemiluminescent moiety to the particle is due to the binding of the analyte. If no analyte is present, the particle (bead) will not emit any light. If the analyte is present, the bead will carry chemiluminescent moiety through the binding of analyte on it and will emit fluorescent light excited by the chemiluminescent moiety. Since the fluorescent particle is colour/size coded, its ID can be determined and the light intensity indicate the amount of analyte in the sample. In this case, no laser is needed. Because the chemiluminescent light is weaker than laser, the flow speed needs to be decreased to increase the detection time.

Alternatively, using the chemiluminescent binding pair-analyte-fluorescent particle sandwich structure, the detection of the analyte is based on the CL and the bead ID is determined by laser excitation; and the detection of chemiluminescent light and fluorescent light can be done sequentially and space separated to eliminate the fluorescent light interfering the CL light. For example, the bead pass through the first detection window without laser illuminating, the detection is for the CL light to determine the analyte binding. When the bead passes though the next detection window the laser/lasers are used to illuminate the bead to determine the bead ID. Because the laser is much stronger than the CL light and the CL light can be selected not to excite the fluorescent bead, the bead ID determination will not be interfered.

The current inventions also disclose a homogenous chemiluminescent assay involving the CL enhancer-producing enzyme. One of the binding pair is CL enhancer-producing enzyme-affinity group conjugate and the second binding pair is the peroxidase-affinity group conjugate. This pair can form a sandwich structure of CL enhancer-producing enzyme-affinity group conjugate—analyte—peroxidase—affinity group conjugate at the presence of the analyte. Because the enhancer-producing enzyme and peroxidase are close to each other due to analyte binding, the substrate of the enhancer-producing enzyme (pro enhancer) will become the enhancer molecule, which will then be converted to an active form of enhancer (which can trigger/enhance the CL reaction of a chemiluminescent compound) by the peroxidase. When the chemiluminescent compound is present (e.g. luminol, acridan), it will emit light indicating the presence of the analyte. Suitable enhancer include but not limited to the enhancer for luminol or acridan/peroxidase system, such as those described in U.S. Pat. No. 6,602,679 and U.S. Pat. No. 5,306,621 (e.g. p-iodophenol, p-hydroxycinnamic acid or p-imidazol-1-ylphenol, p-phenylphenol). The pro enhancer can be any molecule that can be converted to an enhancer molecule by certain enhancer-producing enzyme. For example, p-iodophenol phosphate for alkaline phosphatase to generate p-iodophenol; p-iodophenol beta-galactoside for beta-galactosidase. Many more can be found in US patent U.S. Pat. No. 5,306,621. The chemiluminescent compound can be any molecule that can emit light by the trigger/enhancement of the enhancer (e.g. luminol, isoluminol, TCPO, acridan used in peroxidase chemiluminescent system). One can also add some CL inhibitor such as scavengers of reactive oxygen and free radicals (e.g. p-nitrophenol or SOD or those described above) to the assay mix to reduce the background caused by the CL from none binding enhancer-producing enzyme and none binding peroxidase. Compound/molecules (e.g. heme, gold nano particle and more described previously) that can function like peroxidase can also be used as peroxidase instead.

In example 6, the assay is to detect the peptide human chronic gonadotropin (hCG) in the serum sample. All the reagents can also be added together at the beginning. Either set horseradish peroxidase/antibodyl or set alkaline phosphatase/antibody 2 can be immobilized on solid phase. These two set can also be in the form of particle (either one of them or both). Detergents or polymeric surfactants (e.g. Polymeric cationic surfactants including those comprising quaternary ammonium and phosphonium salt groups or onium groups) or CL enhancer (such as ammonium, cationic homopolymer or copolymer comprising positively charged onium groups, BDMQ, TMQ, TBQ, TPQ, TB, Sapphire, Sapphire 11, THQ, phosphonium polymers, and/or copolymers of ammonium and/or phosphonium monomers or the like) can be coupled with either alkaline phosphatase-antibody conjugate or horseradish peroxidase-antibody conjugate or both; or co immobilized with them on solid phase.

The alkaline phosphatase-antibody conjugate or horseradish peroxidase-antibody conjugate or both can also be coupled with a fluorescent moiety which can be excited by luminol chemiluminescent light and therefore the detection will be for the fluorescence light. As described above, using different fluorescent moiety can allow multiplex detection. A luminol CL inhibitor p-nitrophenol or SOD can also be added to the assay mix to reduce the background caused by the non-binding alkaline phosphatase and horseradish peroxidase.

It is known that gold nanoparticles (e.g. those described in Anal. Chem. 2005. 77, 3324-3329; Biosensors and Bioelectronics 24 (2009) 3581-3586) at the presence of peroxide compound such as H2O2 can trigger or inhibit (J. Phys. Chem. C 2007, 111, 4561-4566) the chemiluminescent of luminol, isoluminol and acridan or the like or other molecules that can emit light at the presence of peroxidase and peroxide. The small size gold nanoparticles (e.g. diameter <5 nm) would inhibit the chemiluminescence and larger size gold nanoparticles (e.g. >10 nm) can enhance the chemiluminescence. The current invention discloses a homogenous assay to detect analyte using affinity group labeled gold nanoparticles. Examples of affinity group include antibody, antigen, aptamers and etc. For example, the gold nanoparticles can be labeled with none mono clone antibody against certain antigen to detect antigen or be labeled with certain antigen to detect certain antibody. The labeling of gold nano particle is well known to the skilled in the art. Therefore the sandwich structure even the agglutination network structure will form which contain more than one gold nano particles. Because the formed multiple gold nano particle clusters have different catalytic capacity for the chemiluminescent reaction, the change in the intensity of chemiluminescent light (CL) can be used to determine the presence of the analyte when the chemiluminescent compound (e.g. luminol, acridan, peroxymonocarbonate-eosin Y system, luminol-K3Fe(CN)6 system, ABEI, TCPO, luminol-AgNO3 system) and sometimes oxidant or peroxide too (or molecules that can trigger the CL, e.g. hydrazine for luminal, potassium permanganate or NaIO4) is provided. For some affinity groups, gold particle size and analyte type the CL will be higher when more analyte is present while other affinity groups, gold particle size and analyte type the CL will be lower. One can establish a standard curve of CL for certain analyte, gold particle size and certain affinity groups and use it for the assay to determine the concentration of the analyte in the sample; and once the analyte amount vs CL intensity curve is established, it can be used to determine the amount of analyte in a sample based on the CL generated from this sample. High salt (e.g. 0.5 M NaCl) can also be added before the CL reaction.

In example 7, the assay is to detect the peptide human chronic gonadotropin (hCG) in the serum sample.

There are many gold particle agglutination assay/colloidal gold agglutination assay which use color change or light absorbing to detect analyte, one can easily convert these assays to chemiluminescent assay by adding chemiluminescent compounds (e.g. luminol and H2O2) with/without high salt in to the assay to convert it into a CL assay.

Yet another format is to use none cross-linking aggregation of gold nano particle (e.g. described in ChemBioChem 2008, 9, 2363-2371, preferably 5˜100 nm size). The gold nano particle is covalently labeled with an affinity group (e.g. nucleic acid probe, aptamer or antibody); which preferably only allow mono valent binding (only one affinity group bind with one analyte therefore will not have cross linking between gold particles). The affinity group can also be immobilized on gold nano particle none-covalently though linker/carrier which is covalently coupled with the gold surface (e.g. a biotin labeled DNA probe in coupled to a 15 nm avidin coated gold particle through the biotin-avidin binding). When the analyte is present, it will bind with the affinity group on the gold particle and therefore change the aggregation behavior of the particle in certain condition (e.g. high salt) therefore change its CL catalytic capacity, therefore with added CL reagent (e.g. luminol and H2O2) and aggregation triggering reagent (e.g. high salt) the analyte will change the CL intensity. The change in CL intensity will tell the presence of the analyte. Different affinity group binding with different analyte may affect the aggregation of gold nano particle differently. Some binding may enhance the aggregation while some may inhibit the aggregation. Normally the aggregation caused by gold nano particle will enhance its catalytic capability for CL. The aggregation condition also varies. For example when high salt is used to induce aggregation, the salt species and concentration need to be determined experimentally. For example, one can first start from NaCl 0.1 M then gradually increase the concentration to see if it induces the aggregation. Once the concentration is determined (e.g. it can cause the gold particle binding with analyte aggregate but the none binding gold particle not aggregate or the gold particle binding with analyte not aggregate but the none binding gold particle aggregate); this concentration can be used in the assay. The aggregation-triggering step (e.g. adding high salt) can be either after or at the same time with the binding with analyte step. Then the CL substrate (e.g. luminol and H2O2) is added and the CL is measured. One embodiment is described in example 8.

In other examples aptamers are used to coat the gold nano particle. The analyte binding aptamer coated on the gold nano particle can be used to detect the analyte. In example 9, the adenosine-binding DNA aptamer Au-T10Ado coated gold particle described in J. Am. Chem. Soc., 2008, 130 (11), pp 3610-3618 is used to detect adenosine.

Yet another format is to use none catalytic particle such as latex beads instead of gold nano particle but immobilize CL generating or inhibiting enzyme and affinity groups on it, a format similar to the latex bead agglutination assay. When the beads forming binding clusters due the analyte binding, the enzymes will be very close therefore have lower catalytic capacity due to the local competing for substrate, therefore the CL intensity will also be changed. For example, latex micro spheres labeled with peroxidase and antibody can be used to detect antigen by adding luminol/H2O2/p-iodophenol or acridan/H2O2/p-iodophenol; the more the antigen, the lower the CL. Similarly, latex micro spheres labeled with alkaline phosphatase and antibody can be used in combination with 1,2 dioxetane AP substrate. If latex micro spheres labeled with ATPs (enzyme that hydrolysis ATP) and antibody are used to detect antigen by adding firefly luciferase/ATP/firefly luciferin, the more the light, the higher the CL.

The current inventions also disclose a method used for the homogenous chemiluminescent or fluorescent assay cited or described in the current inventions. In these assays, when the fluorescence or CL for detection is localized in a layer of surface (e.g. the light emitting generated from analyte binding is on the surface the microwell plate); and the detector or light collecting device for the detector (e.g. window, minor, optic fiber) is placed close to the light emitting layer and far away to most of the background light such as the light generated from none analyte binding (e.g. the detector is placed close to the bottom of the transparent microwell plate instead of on top of the microwell plate); a light absorbing/blocking reagent can be added to the assay mix to reduce these back ground light and therefore increase the relative detection for the analyte. The reason is for example, the fluorescence or CL for detection is generated from the bottom surface of the transparent microwell plate because the affinity group is coated on the bottle of the microwell, when the detector is placed under the well, the light generated from the bottle surface will be absorbed less by the a light absorbing reagent added because the light will encounter less light absorbing reagent since the light source is close to the surface, while the light generated from other part (so it is not caused by analyte binding) such as those from the upper part of the solution in the well will have to travel a much longer distance in the assay mix before it reach the detector therefore it will have a much bigger chance to encounter the a light absorbing molecule and be absorbed.

The principle is to add certain compounds or particles that can absorb the light for detection; preferably they will have high abs for the wavelength of the light for detection. They can be light blocking reagent instead of light absorbing reagent. Examples of them include dye, pigment, and quencher, light absorbing particles, light reflecting particles. It can be done by adding one species of them or by adding the combination of several species (e.g. several dye). Because different dye/pigment/quencher has different absorption coefficient for different wavelength, the concentration (and combination if use multiple dye/pigment/quencher) of them in different application need to be adjusted accordingly to reach the best signal noise ratio. One can start from the concentration that almost completely absorb light generated then decrease the concentration until it does not or only slightly decrease light generated from analyte binding but greatly decrease the light generated not from analyte binding. For example, if the light for detection (not a fluorescence type assay) is from the captured renilla luciferase on microwell surface forming analyte binding sandwich structure, McCormick food coloring red or yellow dye can be added at the concentration of 0.005%˜0.0005%. QSY-35 quencher (abs max 470 nm) can also be used. When acridinium (Em 420 nm) is the direct light source for detection, DABCYL acid quencher can be added since it has high abs at 425 nm. When firefly luciferase is the direct light source for detection, QSY-9 quencher (abs max 562 nm) can be used. If QD 650 is the final light source for detection, one can add a dye having high abs at 650 nm to reduce the background. If light for detection is generated not from a surface, e.g. the final chemiluminescent or fluorescent sandwich structure is not captured on a layer of surface (e.g. they are evenly distributed in the assay solution), this method will not work. Particles can absorb or block the light can also be used. For example, magnetic micro particles containing high content of Fe oxide can also be added, because they have dark color and are not transparent, they can absorb and block the light nearby since they can suspend in the solution. Another choice is fine carbon powder; they can also absorb light. Further more they can absorb the molecules that can generate CL or fluorescence but not bind with analyte therefore reduce their light generating capability. Other absorbent that can absorb the molecules that generating back ground light can also be added. For example, amine coated beads can bind with firefly luciferin, therefore if the assay use firefly luciferase-affinity group—analyte—luciferin generating enzyme-affinity group, this beads can be added to reduce the back ground.

In one example, the assay is performed based on the Example 12 of 20070264664 application, the modification is that the luminomter detector window is placed right underneath the well to detect the bottom of the well only and McCormick food coloring red dye is added to reach the concentration of 0.01%.

Using dark magnetic micro particles can also help the assay speed by providing string/mixing effect to the assay solution if a rotating/moving magnetic field is applied to the assay solution because the magnetic beads will keep on moving to function as a internal shaker/mixer. If the detection is focused on the bottom surface of the well, a magnetic field can be applied to guide the magnetic micro particles to form a layer to cover the bottom surface therefore block the light form the solution above, which is the background light. Dye/pigment can also be coated on or trapped in the micro particles to enhance their light absorbing/blocking capability. None magnetic light absorbing/blocking particles can also be used by mechanical means such as using gravity or centrifuge to form a light-blocking layer to function as the same.

The analyte binding and therefore the light emitting can also be placed on magnetic microparticle instead on the surface of the microwell. In the detection step, a magnetic filed is need to be applied to move the magnetic particle close to the detector window. In one example, the assay is performed based on the Example 14 of 20070264664 application, the modification is that the luminomter detector window is placed right underneath the well and a magnetic filed is applied to enable the magnetic particle to form a layer at the bottom of the well before the detection. It is preferred that the formed magnetic particle layer covers all the bottom of the well or cover all the area of the window of the detector. Here the dark color magnetic particle itself function as a light blocking for the light from upper layer solution. McCormick food coloring red dye can also be added at the concentration of 0.005% to further reduce the background. Other light absorbing/blocking beads can also be used as solid support as long as they can form a light-blocking layer by certain means. Other nano/microparticle can also be used as solid support as long as they can form a light-generating layer by certain means (e.g. centrifuge); if the particles can absorb/block light, a dye may not be needed to add to the assay mix; if the particle is transparent, the light absorbing/blocking reagent will be needed in the assay solution.

Optic fiber (e.g. those described in Analytical Biochemistry 282, 142-146, 2000) is also a suitable solid phase for the assays described in the current inventions. The affinity groups will be immobilized on the tip end of fiber or immobilized on the surface of microsphere and then the bead is placed on the distal tip of the fiber. Therfore the CL and/or fluorescence due to analyte binding will take place on the tip of the fiber. Using fiber bundle that each fiber detects different analyte the fiber-optic array can be made for multiplex detection. Light absorbing reagent can also be added to further reduce the background. However in many applications adding light absorbing reagent is not necessary since optic fiber only directionally collect light. The optic fiber itself can also be surrounded by none transparent coating to reduce the light coming in from none distal tip.

For example, ATP generating enzyme and antibody are co immobilized on tip end of the fiber; the firefly luciferase-antibody conjugate will bind to the fiber end by the binding with the analyte. When ATP generating enzyme substrate and firefly luciferin is present, the fiber will receive a signal light for detection. In another format, renilla luciferase and antibody for analyte are co immobilized on the end of the fiber, QD 650 is also coated with antibody for analyte, when the analyte is present, the QD will bind on the fiber tip and the fiber will pick up the QD fluorescent light excited by luciferase for detection.

In example 10, the assay is to detect the peptide human chronic gonadotropin (hCG) in the serum sample. If horseradish peroxidase-antibody conjugate or gold nanoparticle (36 nm)-antibody conjugate is used instead of alkaline phosphatase-antibody conjugate to coat the solid phase surface, acidan or luminol in H2O2 with p-iodophenol enhancer can be added instead of 1,2-dioxetane AP substrate and Sapphire enhancer. The horseradish peroxidase-antibody conjugate or alkaline phosphatase-antibody conjugate can also be coated on the optic fiber end instead of micro well plate.

The light absorbing/blocking reagent+detector positioning method described above can also be used in the current chemiluminescent type immunoassay or nucleic acid binding assay to reduce the number of washing steps or completely eliminate the washing step. For example, in the 22-plex chemiluminescent micro array for Pneumococcal Antibodies described in Am J Clin Pathol 2007; 128:23-31; Plates were incubated for 30 minutes at 37° C. and washed 3 times. After addition of peroxidase-conjugated antihuman IgG to each well, plates were incubated again for 30 minutes at 37° C. and washed 5 times with Tris-buffered saline containing 0.05% Tween 20. The assay protocol can be modified as following based on the current invention, which is described in example 11.

In example 12, the assay is to detect the peptide human chronic gonadotropin (hCG) in the serum sample. If horseradish peroxidase-antibody conjugate or alkaline phosphatase-antibody conjugate is used instead of luciferase-antibody conjugate to bind with analyte, acidan or luminol in H2O2 or 1,2-dioxetane AP substrate and Sapphire enhancer can be added instead of adding coelenterazine. The horseradish peroxidase-antibody conjugate or alkaline phosphatase-antibody conjugate can also be coated on the optic fiber end instead of micro well plate. Alternatively, electrochemiluminescence (ECL) reagent (e.g. those described in Talanta 78 (2009) 399-404) can also be used to from the sandwich-type immuno complex. These assays can also be readily converted to the hybridization based nucleic acid test.

### EXAMPLES

### Example 1

Horseradish peroxidase is conjugated to the oligonucleotide probe P1. P1 is a 20 mer oligonucleotide having a linker with an amine group at 5′ end, which can be used to couple with the enzyme. The carboxy-activated QD705 is conjugated to the oligonucleotide probe CP1; CP1 is a 20 mer oligonucleotide complimentary to P1 with an amine linker at 3′ end for the coupling. The assay is performed using 4.0 pmol of peroxidase-P1 and QD705-CP1 and by changing the amount of the unlabeled target probe P1 (5, 10, 20, 50, 100 and 200 pmol). Probe mixture in 200 μL of hybridization buffer (0.1M NaCl, 50 mM borate buffer pH 8.0 containing 0.1% BSA) is incubated at 37° C. for 30 min and is allowed to cool down to room temperature before the addition of the CL reaction buffer (0.1M sodium borate solution (pH 10.0) containing of 1.0×10⁻⁴ M luminol, 5.00×10⁻⁴ M H2O2 and 5.00×10⁻⁴ M para-iodophenol). Luminescence intensity counts are measured at 460 nm and at 710 nm. The ratio (1460 nm/I710 nm) is plotted against the concentration of the target. One needs to know that this assay is in the competitive binding assay format-an assay based on the competition between a labeled and an unlabelled ligand in the reaction with a receptor-binding agent (e.g. antibody, receptor, nucleic acid). The principle and format of competitive binding assay is well known to the skilled in the art. In the above example, higher the ratio (1460 nm/I710 nm) indicates higher target concentration (lower fluorescence intensity). One can readily convert the above assay into a non-competitive binding assay by change the sequence of the oligonucleotide probe to enable the peroxidase bind with the QD closely. If the affinity groups are antibodies that can form sandwich structure with analyte instead of oligonucleotide probes, the above assay will become an immunoassay.

### Example 2

The detection solution contains the following:

| Imidazole (pH 7.0) | 50 | mM |
|---|---|---|
| BSA | 1 | mg/mL |
| ATP, Na Salt | 12 | mM |
| DTT | 10 | mM |
| Co-enzyme A | 1 mM or 10 mg/mL | |
| MgSO4 | 15 | mM |
| CaCl2 | 4 | mM |
| Mannitol | 1% | |
| Sucrose | 0.1% | |
| Firefly Luciferase- | 2 | μg/mL |
| antibody conjugate | | |
| QD-antibody conjugate | 6 | μg/mL |

100 microliters of assay solution described above is mixed with 100 microliters of HCG containing sample and incubated at 25 degree C. for 20 minutes. Next, 100 microliters of firefly luciferin in 0.01M PBS buffer (5 ug/mL) solution is added to the reaction mix and placed in a luminometer for reading the light signal at >700 nm. The higher the light signal reading, the higher the HCG concentration is in the sample. The luciferase and affinity groups can also be coated on microparticles or nano particles as described above.

The luciferase can be used is also not limited to firefly luciferase. For example, click beetles luciferase using firefly luciferin, bacterial luciferase using FMNH₂, cypridina luciferase using cypridina luciferin and renilla luciferase using coelenterazine can also be used instead. However when different luciferase is used, their specific luciferin and CL condition need to be provided in the assay mix. For example, if 5 ug/ml renilla luciferase-antibody conjugate is used in the above assay, coelenterazine (1 μL of 1 mg/mL) can be added to the assay to trigger the light emitting and ATP/firefly luciferin is not needed.

### Example 3

The first monoclonal antibody for human chronic gonadotropin (hCG) is linked with alkaline phosphatase, the second monoclonal antibody for hCG, which recognizes a different portion of the hCG molecule other than that recognized by the first monoclonal antibody is linked with firefly luciferase, Rhodamine Red (Ex570/Em590) is also coupled to the second monoclonal antibody.

The detection solution contains the following:

| Imidazole (pH 8.0) | 50 | mM |
|---|---|---|
| BSA | 1 | mg/mL |
| ATP, Na Salt | 12 | mM |
| DTT | 10 | mM |
| Co-enzyme A | 1 mM or 10 mg/mL | |
| MgSO4 | 15 | mM |
| CaCl2 | 4 | mM |
| Mannitol | 1% | |
| Sucrose | 0.5% | |
| Sodium Azide | 0.05% | |
| Firefly Luciferase-antibody- | 2 | μg/mL |
| Rhodamine Red conjugate | | |
| alkaline phosphatase - | 2 | μg/mL |
| antibody conjugate | | |

100 microliters of assay solution described above is mixed with 100 microliters of HCG containing sample and incubated at 25 degree C. for 20 minutes. Next, 50 microliters of substrate (firefly luciferin 0-phosphate, 50 micrograms/mL in 50 mM imidazole buffer (pH 8.0)) solution is added to the reaction mix and placed in a luminometer for reading the light signal of 590 nm. It is preferred that an antibody against firefly luciferin is included in the detection mix to increase the signal to noise ratio. ATP producing enzyme such as sulfate adenylyltransferase (ATPS), which converts APS (adenosine 5′-phosphosulfate) to ATP in the presence of PPi can also be used instead of alkaline phosphatase as described in U.S. patent application Ser. No. 12/287,916 and free luciferin need to be in the assay mix; the assay condition can be readily adapted accordingly. Other fluorescent moiety such as QD 650 or fluorescent microparticle can also be used to immobilize both the firefly luciferase and the second antibody instead of using Rhodamine Red. If renilla luciferase and QD 650 is used, coelenterazine O-phosphate (mono or di) should be used instead of firefly luciferin 0-phosphate and no ATP is needed.

### Example 4

The first monoclonal antibody (1^{st} affinity group) for human chronic gonadotropin (hCG) is coated on Eu oxide nano particle, the second monoclonal antibody (2^{nd} affinity group) for hCG, which recognizes a different portion of the hCG molecule than that recognized by the first monoclonal antibody is coupled with alkaline phosphatase.

The detection solution contains the following:

| | | |
|---|---|---|
| PBS (pH 8.5) | 50 | mM |
| BSA | 1 | mg/mL |
| alkaline phosphatase - | 2 | μg/mL |
| antibody conjugate | | |
| Eu oxide-antibody conjugate | 6 | μg/mL |

100 microliters of assay solution described above is mixed with 100 microliters of HCG containing sample and incubated at 25 degree C. for 20 minutes. Next, 100 microliters of 1,2-dioxetane AP substrate in 0.01M PBS buffer, 5 ug/mL and suitable amount of Sapphire enhancer solution is added to the reaction mix and placed in a luminomter for reading the light signal at >600 nm.

### Example 5

The first monoclonal antibody (1^{st} affinity group) for human chronic gonadotropin (hCG) is coated on lum size carboxylate polystyrene micro particle which is then coated with dioxetane enhancer, the second monoclonal antibody (2^{nd} affinity group) for hCG, which recognizes a different portion of the hCG molecule than that recognized by the first monoclonal antibody is coupled with alkaline phosphatase.

The detection solution contains the following:

| | | |
|---|---|---|
| PBS buffer (pH 8.5) | 50 | mM |
| BSA | 1 | mg/mL |
| alkaline phosphatase - | 2 | μg/mL |
| antibody conjugate | | |
| enhancer particle - | 6 | μg/mL |
| antibody conjugate | | |

100 microliters of assay solution described above is mixed with 100 microliters of HCG containing sample and incubated at 25 degree C. for 20 minutes. Next, 100 microliters of 1,2-dioxetane AP substrate in 0.01M PBS buffer, 5 ug/mL is added to the reaction mix and placed in a luminomter for reading the light signal.

### Example 6

The first monoclonal antibody (1^{st} affinity group) for human chronic gonadotropin (hCG) is coupled with horseradish peroxidase, the second monoclonal antibody (2^{nd} affinity group) for hCG, which recognizes a different portion of the hCG molecule than that recognized by the first monoclonal antibody is coupled with alkaline phosphatase.

The assay solution contains the following:

| | | |
|---|---|---|
| PBS (pH 8.5) | 50 | mM |
| BSA | 1 | mg/mL |
| alkaline phosphatase - | 2 | μg/mL |
| antibody conjugate | | |
| horseradish peroxidase - | 2 | μg/mL |
| antibody conjugate | | |

100 microliters of assay solution described above is mixed with 100 microliters of HCG containing sample and incubated at 25 degree C. for 20 minutes. Next, 100 microliters of 0.1M sodium borate solution (pH 9.0) containing of 1.0×10⁻⁴ M luminol, 5.00×10 M H2O2 and 5.00×10⁻⁴ M p-iodophenol phosphate is added to the reaction mix and placed in a luminomter for reading.

### Example 7

The first monoclonal antibody (1^{st} affinity group) for human chronic gonadotropin (hCG) is coupled with gold nano particles (5 or 10 nm), the second monoclonal antibody (2^{nd} affinity group) for hCG, which recognizes a different portion of the hCG molecule than that recognized by the first monoclonal antibody is also coupled with gold nano particles (5 or 10 nm). These two gold nano particles are mixed together at equal ratio.

The assay solution contains the following:

| | | |
|---|---|---|
| PBS (pH 8.5) | 50 | mM |
| BSA | 1 | mg/mL |
| Gold nano particle - | 5 | μg/mL |
| antibody conjugate | | |

100 microliters of assay solution described above is mixed with 100 microliters of HCG containing sample and incubated at 25 degree C. for 20 minutes. Next, 100 microliters of 0.1M sodium borate solution (pH 10.0) containing of 5.0×10⁻⁴ M luminol, 5.00×10⁻⁴ M H2O2 is added to the reaction mix and placed in a luminomter for reading. A optionally 200 ul 1M NaCl can also be added and incubated for 5 min before adding luminol and H2O2. 2×10⁻⁵M hydrazine can also be used instead of H2O2 to initiate luminol chemiluminescence. Once can establish a CL intensity vs HCG concentration curve by testing samples containing series amount of HCG and use this curve to determine the HCG amount in a unknown sample. Alternatively, other metal particle that can catalyze the CL reaction such as platinum colloids can also be used instead of the gold nano particle.

### Example 8

In order to detect a 20 mer oligonucleotide as target, a 5′-SH linker containing 20 mer oligonucleotide probe complimentary to this target oligonucleotide is covalently labeled to 13 nm gold particles, preferably each gold particle carry less than 10 copies of probe, more preferably carry less than 5 copies. In order to detect the target in a sample, 100 uL of above gold particle (5 ug/ml) in 0.1 M PBS pH 8 is mixed with 50 ul sample solution and incubated at 25 degree C. for 10 minutes. Next, 300 uL of high salt (e.g. NaCl 0.2-0.6 M) is added and incubate for 2 min, then 100 uL 0.1M sodium borate solution (pH 10) containing of 5×10⁻⁴ M luminol, 5×10⁻⁴ M H2O2 is added to the reaction mix and placed in a luminomter for reading. Alternatively, high concentration salt can also be added at the beginning of hybridization. The more the target, the higher the CL is.

### Example 9

The assay is performed as following: assay solutions (250 μL) contained target adenosine, Au-T10Ado (˜3 nM), Tris-HCl (20 mM), pH=7.5, NaCl (100 mM) and MgCl2 (60 mM). Specifically, a mixture (180 μL) of Au-T10Ado (4.14 nM), Tris-HCl (28 mM) and NaCl (138 mM) is first prepared. A solution (70 μL) of MgCl2 (220 mM) and adenosine are then added for 10 min at room temperature. 50 uL 0.1M sodium borate solution (pH 10) containing of 5.0×10⁻⁴ M luminol, 5.0×10⁻⁴ M H2O2 is added to the reaction mix and placed in a luminomter for reading. The more the target, the lower the CL is.

### Example 10

The first monoclonal antibody (1^{st} affinity group) for human chronic gonadotropin (hCG) is coupled with QD 650, the second monoclonal antibody (2^{nd} affinity group) for hCG, which recognizes a different portion of the hCG molecule than that recognized by the first monoclonal antibody is also coupled with alkaline phosphatase. The alkaline phosphatase-antibody conjugate is immobilized on polystyrene micro well surface. 200 uL of PBS (pH 8.5) 50 mM; BSA 1 mg/mL, QD 650-antibody conjugate 6 μg/mL is added to microwell, the sample containing hCG is also added and incubated at 25 degree C. for 20 minutes. Next, 50 uL of 1,2-dioxetane AP substrate in 0.01M PBS buffer, 5 ug/mL containing Sapphire enhancer and McCormick blue food coloring dye (or QSY-21 dye) 5 ug/mL is added to the reaction mix. The detection window is facing the bottom of the well to collect the light signal at >600 nm.

### Example 11

Plates described in the reference is incubated for 30 minutes at 37° C. and washed 3 times. After addition of peroxidase-conjugated antihuman IgG to each well, plates is incubated again for 30 minutes at 37° C. and without further washing, 10 uL McCormick (aka Schilling) food coloring red dye is added to reach the concentration of 0.01% and the detector is placed under the well. The later steps are the same of those in the reference. Alternatively, peroxidase-conjugated antihuman IgG can also be added at the beginning and no washing step is performed before the CL reaction. Therefore the assay becomes a true homogenous (none washing) multiplex assay.

### Example 12

The first monoclonal antibody (1^{st} affinity group) for human chronic gonadotropin (hCG) is coated to the micro well plate, the second monoclonal antibody (2^{nd} affinity group) for hCG, which recognizes a different portion of the hCG molecule than that recognized by the first monoclonal antibody is coupled with renilla luciferase. 200 uL of PBS (pH 8.5) 50 mM; BSA 1 mg/mL, renilla luciferase-antibody conjugate 1 μg/mL is added to microwell, the sample containing hCG is also added and incubated at 25 degree C. for 20 minutes. Next, coelenterazine (1 μL of 1 mg/mL) is added and the detection window is facing the bottom of the well for the detection. McCormick food coloring red or yellow dye will be added to reach the final concentration of 0.005%˜0.0005%. QSY-35 quencher (abs max 470 nm) can also be used.

What is claimed is:

1. A method to detecting analyte in a sample, comprising:
a) contacting said sample with a first ligand of the analyte coupled with luminescence producing enzyme and a second ligand of the analyte coupled with fluorescent producing moiety, and
b) detecting the light generated from said fluorecence.

2. A method to detecting analyte in a sample, comprising:
a) contacting said sample with a ligand of the analyte coupled with gold nanoparticle, and
b) adding chemiluminescent reagent to the sample, and
c) detecting the light generated from gold nanoparticle triggered luminescence.
